(19) United States
(12) Patent Application Publication
(10) Pub. No.: US 2015/0194817 A1
(43) Pub. Date: Jul. 9, 2015

(54) INTEGRATED DEVICES FOR LOW POWER QUANTITATIVE MEASUREMENTS

(71) Applicant: MC10, Inc., Cambridge, MA (US)

(72) Inventors: Stephen Lee, Cambridge, MA (US); Roozbeh Ghaffari, Cambridge, MA (US)

(21) Appl. No.: 14/588,765

(22) Filed: Jan. 2, 2015

Related U.S. Application Data
(60) Provisional application No. 61/923,420, filed on Jan. 3, 2014.

(51) Int. Cl. H02J 5/00; G01N 33/49; G01N 33/66; G01N 33/00

(52) U.S. Cl. CPC H02J 5/005; G01N 33/00; G01N 33/49; G01N 33/66

(57) ABSTRACT

A device includes a wirelessly enabled energy harvesting device, an energy storage component, a DC-DC converter, and a functional circuit. The energy storage component is electrically coupled to the wirelessly enabled energy harvesting device for storing energy harvested by the wirelessly enabled energy harvesting device from a wireless transmitting device positioned adjacent to the device. The DC-DC converter is electrically coupled to the energy storage component for receiving a voltage output from the energy storage component and converting the received voltage output to a second voltage level to provide power to one or more components of the device. The functional circuit is for measuring a concentration of a substance in a fluid sample. The functional circuit is coupled to the DC-DC converter such that the functional circuit obtains at least a portion of the power provided by the DC-DC converter.

### CROSS-REFERENCE TO RELATED APPLICATIONS

This application claims the benefit of U.S. Provisional Application No. 61/923,420, filed Jan. 3, 2014, which is hereby incorporated by reference herein in its entirety.

### FIELD OF THE INVENTION

The present disclosure relates generally to a sensor system, and more particularly to a sensor system that is powered in proximity to a wireless device and allows various sensors to be powered based on the retained power from the wireless device.

### BACKGROUND OF THE INVENTION

Existing measurement devices for performing quantitative measurements can be bulky, due to the size of the power source needed to power operation of the measurement device. The relatively bulky size of the existing measurement devices can limit the applicability of such measurement devices. Further, the size of the power source not only adds bulk to the existing measurement devices, but also restricts possible arrangements of components within the existing measurement devices. Thus, modifying the dimensions of prior measurement devices is inhibited. The present disclosure is directed to solutions for these and other problems.

### SUMMARY OF THE INVENTION

Ultra-small devices (e.g., wearable devices) are constrained in size by the battery of the device. Advances in semiconductor processes allow for measurement and action (e.g., delivery of therapy), but power requirements limit the application of such devices in small or thin form factors. The present disclosure includes description of a technique that mitigates the problem of batteries on ultra-small devices and enables application of semiconductor technologies in small form factors (e.g., thin, flexible, stretchable, wearable devices that confirm to a user's skin). The present disclosure utilizes carefully selected and specifically designed hardware and empirically tested software to control the timing of low power electronics such that the low powered electronics can draw an acceptably low current from either (1) an energy harvesting device such as, for example, an NFC device (e.g., an NFC EEPROM), a solar device, a thermoelectric device and/or a (2) a small battery that meets the required form factor.

According to some implementations, a device (e.g., device 720, 800, 1000) includes electronic components (e.g., 902, 1060, 230), which are selected so that they consume minimal power yet meet the application's requirements. Although the electronic components of the device (e.g., 902, 230 and 1060) are low power devices, when they turn on, they may draw excessive current that will cause an energy harvesting device (e.g., wirelessly enabled energy harvesting device 210, 1010) or a small battery (e.g., battery 910) to collapse. This typically happens at startup or it may happen during certain stages of measurement (e.g., due to power drawn by a sensor (e.g., sensor 1070) during measurement/testing).

According to some implementations, to solve the problem of power draw at startup, a pre-charge circuit (e.g., pre-charge circuit 901, 1052) with timing control circuitry (e.g., timing control circuit 1054) is included in devices of the present disclosure.

According to some implementations, to solve the problem of maintaining steady power during operation, particularly during sensitive measurements (e.g., using the sensor 1070), an MCU (e.g., MCU 903, 1062) is included to control when certain sub-circuits (e.g., memory 1064, ADC 1066, DAC 1068, and sensor 1070) activate and how long they stay on. These sub0circuits can be cycled on and off and used only when needed by the device (e.g., device 720, 800, 1000.

According to some implementations of the present disclosure, a measurement device includes a near-field communication (NFC) enabled energy harvesting device, an energy storage component, a DC-DC converter, a counter, and a functional circuit. The energy storage component is electrically coupled to the NFC enabled energy harvesting device for storing energy harvested by the NFC enabled energy harvesting device from an NFC transmitting device positioned adjacent to the measurement device. The DC-DC converter is electrically coupled to the energy storage component. The functional circuit is electrically coupled to the DC-DC converter. The energy storage component harvests and stores at least a portion of the energy harvested by the NFC enabled energy harvesting device until a first time T₁ set by the counter. The DC-DC converter is activated at a second time T₂ set by the counter using at least a portion of the energy stored in the energy storage component. The functional circuit is activated at a third time T₃ set by the counter using at least a portion of the power provided by the DC-DC converter.

According to some implementations of the present disclosure, a measurement device includes a near-field communication (NFC) enabled energy harvesting device, an energy storage component, a pre-charge circuit, a DC-DC converter, and a functional circuit. The energy storage component is electrically coupled to the NFC enabled energy harvesting device for storing energy harvested by the NFC enabled energy harvesting device from an NFC transmitting device positioned adjacent to the measurement device. The pre-charge circuit is electrically coupled to the energy storage component. The DC-DC converter is electrically coupled to the pre-charge circuit. The functional circuit is electrically coupled to the DC-DC converter. The pre-charge circuit is configured to prevent electrical communication between the energy storage component and the DC-DC converter until the energy storage component stores an amount of energy greater than a threshold energy level and to maintain the electrical communication between the energy storage component and the DC-DC converter thereafter. The functional circuit is configured to activate using at least a portion of the power provided by the DC-DC converter.

According to some implementations of the present disclosure, a measurement device for measuring an analyte in a fluid sample includes a wirelessly enabled energy harvesting device, an energy storage component, a DC-DC converter, and a functional circuit. The energy storage component is electrically coupled to the wirelessly enabled energy harvesting device for storing energy harvested by the wirelessly enabled energy harvesting device from a wireless transmitting device positioned adjacent to the measurement device. The DC-DC converter is electrically coupled to the energy storage component for receiving a voltage output from the energy storage component and converting the received voltage output to a second voltage level to provide power to one or more components of the measurement device. The functional circuit is for measuring a quantity of the analyte in the fluid sample. The functional circuit is coupled to the DC-DC converter such that the functional circuit obtains at least a portion of the power provided by the DC-DC converter.

Additional aspects of the present disclosure will be apparent to those of ordinary skill in the art in view of the detailed description of various implementations, which is made with reference to the drawings, a brief description of which is provided below.

### BRIEF DESCRIPTION OF THE DRAWINGS

The foregoing and other advantages of the disclosure will become apparent upon reading the following detailed description and upon reference to the drawings.

FIG. 1 is a perspective view of batteries according to some implementations of the present disclosure;

FIG. 2 is a schematic view of a circuit diagram of a power circuit including a wirelessly enabled energy harvesting device, an energy storage device, and a DC-DC converter according to some implementations of the present disclosure;

FIG. 3 is a chart illustrating characteristics of a first DC-DC converter according to some implementations of the present disclosure;

FIG. 4 is a chart illustrating characteristics of a second DC-DC converter according to some implementations of the present disclosure;

FIG. 5. is a chart illustrating current load of a measurement device as various sub-systems are turned on according to some implementations of the present disclosure;

FIG. 6 is a flow diagram illustrating a sequence of startup of components of a system according to some implementations of the present disclosure;

FIG. 7 is a flow diagram illustrating step-by-step instructions for placement of a wireless transmitting device relative to a measurement device according to some implementations of the present disclosure;

FIG. 8 is a schematic diagram of a measurement device according to some implementations of the present disclosure;

FIG. 9 is a schematic view of a circuit diagram of a measurement device according to some implementations of the present disclosure; and

FIG. 10 is a schematic view of a circuit diagram of a device according to some implementations of the present disclosure.

While the present disclosure is susceptible to various modifications and alternative forms, specific implementations have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DESCRIPTION OF ILLUSTRATIVE EMBODIMENTS

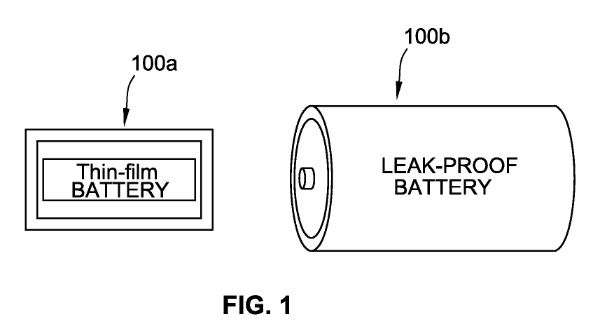

The present disclosure is related to methods, apparatuses, and systems for quantitative analysis using measurement devices that include no power source or a low-power source for such applications as, for example, environmental and/or diagnostic purposes. A low-power source could be a power source providing power lower than about 25 mAH, about 20 mAH, about 15 mAH, about 10 mAH, about 5 mAH, or about 1 mAH. In some implementations, the low-power source could provide lower than about 5 mA peak current, such as but not limited to, a thin-film battery 100a (FIG. 1) with sub-5 mA peak current. The measurement devices of the present disclosure are for detecting and/or quantifying at least one constituent of a sample, such as, but not limited to, a biological sample (e.g., blood, urine, etc.) or other chemical sample.

In some alternative implementations, a measurement device includes a higher-power source, where the higher-power source is maintained dormant or used minimally to replicate the state of a measurement device according to the principles described herein.

The example systems, methods, and apparatus described herein facilitate energy harvesting from computing devices, such as, but not limited to, smartphones for powering data gathering and/or analysis systems.

The example systems, methods, and apparatus described herein also provide innovations in the design of power circuitry, by substantially eliminating the need for an on-board power source. This facilitates many innovative and different designs of the power circuitry of a system.

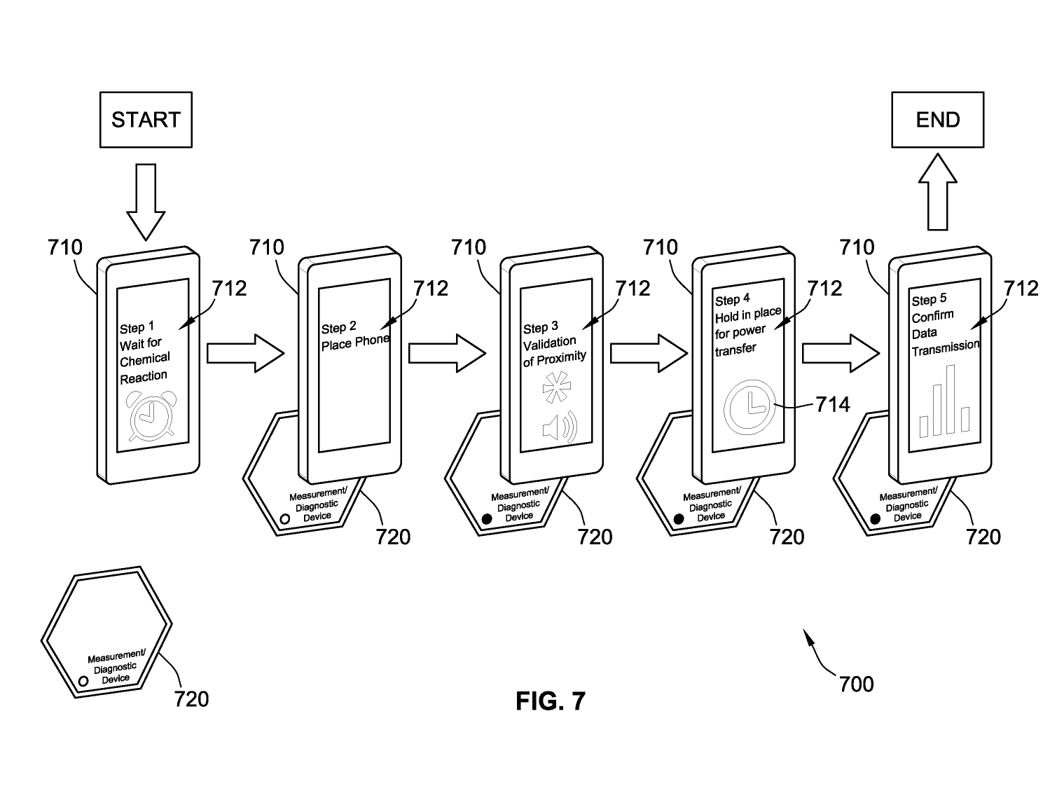

The example systems, methods, and apparatus described herein also provide innovative methods to guide a user to deploy the measurement device in a convenient manner that facilitates energy-harvesting (see e.g., FIG. 7).

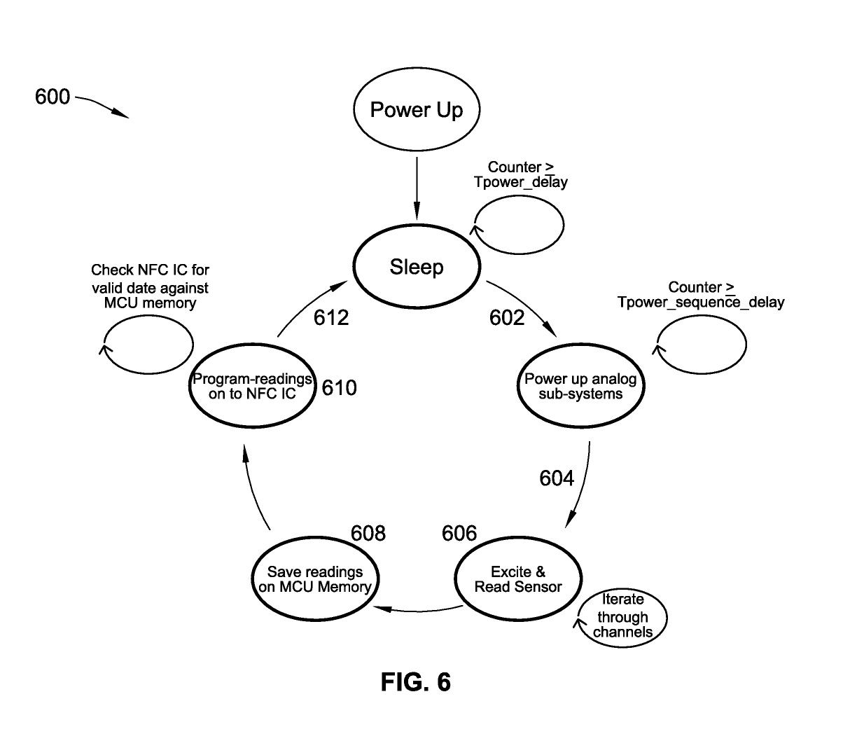

Startup sequences (see e.g., FIG. 6) are described herein that carefully parcel out energy in small quantity to allow full system power. The systems herein may be used for intermittent monitoring applications, where continuous monitoring may not be needed. For example, the systems herein may be used to store harvested energy for a short period of time, sufficient to allow the measurement device to perform data gathering and/or data analysis. In another example, a portion of the stored, harvested energy may be used to perform data storage and/or data transmission.

In some implementations, data may be transmitted to a memory of the system and/or communicated (transmitted) to an external memory or other storage device, a network, and/or an off-board computing device. The external storage device can be a server, including a server in a data center. Non-limiting examples of such a computing device include smartphones, tablets, laptops, slates, e-readers or other electronic reader or hand-held, portable, or wearable computing device, an Xbox®, a WHO, or other game system(s).

Any of the measurement devices according to the systems, methods, and apparatuses described herein may be configured for intermittent use.

Any of the measurement devices according to the systems, methods, and apparatuses described herein may be configured as sensor units, sensor patches, monitoring devices, diagnostic devices, therapy devices, or any other measurement device that can be operated using harvested energy as described herein. As a non-limiting example, the measurement device can be a glucose monitor or other glucose measurement device.

The measurement devices of the present disclosure can be configured for many different types of sensing modalities. Sensing modalities include, for example, detecting and/or quantifying pressure, impedance, capacitance, blood flow and/or the presence of specific substances, such as, but not limited to, chemicals, proteins, or antibodies. In some implementations, the measurement devices are implemented for performing electrical measurement of environmental conditions.

In the field of healthcare, and particularly human diagnostics, point-of-care (POC) testing generally refers to laboratory tests outside of a central laboratory. POC has improved patient care efficiency as it allows diagnostic testing to be performed wherever a patient may be, including in some instance by the patient themselves. POC not only provides the patients with convenience of self-health monitoring, but also allows remote medical record keeping and diagnoses, for example, by uploading the POC test results to a health professional's site through the Internet.

Quantitative information from analysis of a sample can be used for, for example, determining glucose levels or diagnosing diseases (e.g., HIV, malaria, etc.). When a sample, such as but not limited to blood, is placed onto a testing platform, a pre-deposited assay can be used to analyze the sample. As non-limiting examples, a measurement platform based on the example measurement devices described herein can be configured to provide data or other information indicative of at least one constituent of the sample. In an example, the data or other information can be stored to a memory of the testing platform or transmitted wirelessly. In another example, the measurement platform based on the example measurement devices described herein can be configured to provide an indication of the data or other information from the quantitative measurements, such as but not limited to a change in a color indication, a symbol, and/or a digital readout. The results of the quantitative measurements can be used to provide an indication of a condition of an individual, such as but not limited to, a glucose level or an indication of vitamin D level, or a positive or negative indication for an affliction (such as but not limited to HIV or malaria), and/or a degree of progression of an affliction. In some examples, the devices can be configured for performing electrical quantitative measurements that can be used for medical diagnosis, including determining the presences of and/or quantifying, proteins or antibodies, such as but not limited to a malaria diagnosis or a HIV diagnosis.

In some examples, the measurement devices can be configured for performing electrical quantitative measurements for determining dynamic quantities, such as but not limited to blood flow rate or heart rate.

The present disclosure includes various measurement devices with no power source or with a low-power source for use in providing quantitative information relating to a sample or a condition (such as but not limited to an environmental condition or a physiological condition). Such measurement devices include electronic circuitry and processor-executable instructions (including firmware) that facilitate the operation of the measurement device to analyze measurements of a sample or a condition, where the measurement device lacks a power source or includes a low-power source.

According to some implementations, the measurement devices of the present disclosure are a microfluidic test device (e.g., a blood glucose meter) with embedded electronics to acquire a quantized measurement. The microfluidic test device can be configured to transmit quantitative information relating to the sample measured to a computing device (e.g., a smartphone, laptop, desktop computer, etc.).

The measurement device can be configured as a flexible, conformal electronic device with modulated conformality. The control over the conformality allows the generation of measurement devices that can be conformed to the contours of a surface without disruption of the functional or electronic properties of the measurement device. The conformality of the overall conformal device can be controlled and modulated based on the degree of flexibility and/or stretchability of the structure. Non-limiting examples of components of the conformal electronic devices include a processing unit, a memory (such as but not limited to a read-only memory, a flash memory, and/or a random-access memory), an input interface, an output interface, a communication module, a passive circuit component, an active circuit component, etc. The conformal electronic device can include at least one microcontroller and/or other integrated circuit component. The conformal electronic device can include at least one coil, such as, but not limited to, a near-field communication (NFC) enabled coil. Alternatively, the conformal electronic device can include a radio-frequency identification (RFID) component. In some implementations, the conformal electronic devices includes a dynamic NFC/RFID tag integrated circuit with a dual-interface, electrically erasable programmable memory (EEPROM).

The conformal electronic device can be configured with one or more device islands. The arrangement of the device islands can be determined based on, for example, the type of components that are incorporated in the overall conformal device (including the sensor system), the intended dimensions of the overall conformal device, and the intended degree of conformality of the overall conformal device.

As a non-limiting example, the configuration of the one or more device islands can be determined based on the type of overall conformal device to be constructed. For example, the overall conformal device may be a wearable conformal electronics structure, or a passive or active electronic structure that is to be disposed in a flexible and/or stretchable object.

As another non-limiting example, the configuration of the one or more device islands of the measurement device can be determined based on the components to be used in an intended application of the overall measurement device. Example applications include a temperature sensor, a neuro-sensor, a hydration sensor, a heart sensor, a motion sensor, a flow sensor, a pressure sensor, an equipment monitor (e.g., smart equipment), a respiratory rhythm monitor, a skin conductance monitor, an electrical contact, or any combination thereof. In some implementations, the one or more device islands can be configured to include at least one multifunctional sensor, including a temperature, strain, and/or electrophysiological sensor, a combined motion-/heart/neuro-sensor, a combined heart-/temperature-sensor, etc.

In some implementations of the present disclosure, the measurement device is configured without an on-board power source. In such implementations, the degree of conformality of the measurement device is increased relative to a measurements device that includes an on-board power source. Further, the measurement devices disclosed herein can be configured in new form factors allowing the creation of very thin and conformal devices. As a non-limiting example, the average thickness of the measurement device is about 2.5 mm or less, about 2 mm or less, about 1.5 mm or less, about 1 mm or less, about 500 microns or less, about 100 microns or less, or about 75 microns or less. In an example implementation, at least a portion of the measurement device may be folded, or the measurement device may be caused to surround and conform to a portion of a sample to be measured. In an example where at least a portion of the measurement device is folded, the average thickness of the measurement device may be about 5 mm or less, about 4 mm or less, about 3 mm or less, about 2 mm or less, about 1 mm or less, about 200 microns or less, or about 150 microns or less. The lateral, in-plane dimensions can be varied based on the desired application. For example, the lateral dimensions can be on the order of centimeters or fractions of a centimeter. In other examples, the example measurement devices can be configured to have other dimensions, form factors, and/or aspect ratios (e.g., thinner, thicker, wider, narrower, or many other variations).

Non-limiting examples of a computing device applicable to any of the systems, apparatuses, or methods according to the principles disclosed herein include smartphones, tablets, laptops, slates, e-readers or other electronic reader, an Xbox®, a WHO, or other game system(s), or other hand-held or wearable computing device.

As discussed herein, the measurement device can lack a power source or include a power source that provides little power to perform quantitative measurements. As such, the measurement device can be made lower-cost, based on the reduced cost or no cost expended for a power source component, or the avoidance or reduction of costs associated with caring for or charging the power source. Further, the measurement devices can be less complex, due to the fewer or more simplified components in the structure, and as a result could be manufactured in a lower cost fabrication process. Given that the measurement devices may be produced with no power component or a lower-power component, the measurement devices may be better for the environment as there may be fewer chemicals when disposed.

Non-limiting examples of power sources applicable to at least some of the measurement devices of the present disclosure herein include batteries, fuel cells, solar cells, capacitors, and thermoelectric devices. FIG. 1 shows examples of batteries, including bulk low-leakage batteries 100b and thin-film batteries 100a.

In some implementations, the measurement device derives power for performing quantitative measurements through energy harvesting. The energy harvesting component of the measurement device can be any component that may be used to transduce one form of energy to another form of energy (such as but not limited to electrical energy). In some implementations, the measurement device derives power for performing quantitative measurements by energy harvesting from thermal gradients, mechanical vibrations, transverse waves, and/or longitudinal waves. The transverse waves or longitudinal waves may be generated by at least one component of an external computing device (e.g., a smart phone). In some implementations, the energy harvesting component of the measurement device is a metamaterial, an optoelectronic device, a thermoelectric device, a resonator, a coil, or other component that can be configured to couple to a form of energy. The transverse waves may be electromagnetic waves or acoustic waves. The longitudinal waves may be acoustic waves.

In some implementations, the measurement device derives power for performing quantitative measurements by energy harvesting based on radio waves from an external computing device (e.g., a smartphone). In such an implementation, a surface acoustic wave technology may be implemented in the measurement device to exploit a piezoelectric effect to convert the acoustic waver into an electrical signal. For example, the surface acoustic wave sensor may include an interdigitated transducer for the conversion.

In some implementations, the measurement devices of the present disclosure are as single-use devices (e.g., a single use blood glucose test sensor device/system), or devices that can be used for performing two or more quantitative measurements (e.g., a multi-use device, such as, for example, a continuous glucose monitoring sensor device/system maintained in contact with skin of a user). For example, the measurement device may be a re-usable, lower-cost system for quantitative measurements. As a result, the measurement device could provide environmental benefits by, for example, reducing typical waste associated with testing ones blood glucose levels with one-time use test sensors/strips.

In some implementations, the components of the measurement device are arranged such that a specific sequence of activation of the components occurs to minimize the power needs of the measurement device. In some such implementations, the measurement device includes an energy harvesting component and performs quantitative measurements and/or diagnoses as follows. The energy harvesting component of the measurement device harvests power via an external near-field communication (NFC) enabled device (e.g., an NFC coil and/or antenna) at the point of measurement and/or diagnosis. That is, the measurement device performs the measurement and/or diagnosis concurrently with the commencement of the energy harvesting or at any point during the energy harvesting. In this example, the measurement and/or diagnosis can be performed at substantially the same time as the energy harvesting is performed.

In some implementations, the measurement device includes an energy harvesting component and performs quantitative measurements and/or diagnoses as follows. The energy harvesting component of the measurement device harvests power via an external near-field communication (NFC) enabled device, and stores that harvested power in an energy-retaining component of the measurement device. For example, the measurement device can include a capacitive component, and the harvested power can be used to charge the capacitive component. In some examples, the capacitive component can be a low-leakage capacitor or a super capacitor. Non-limiting examples of the low-leakage capacitors applicable to any system or apparatus disclosed herein include an aluminum electrolytic capacitor, an aluminum polymer capacitor, or an ultra-low leakage tantalum capacitor. For some implementations, the aluminum electrolytic capacitor may be a better selection than the ultra-low leakage tantalum capacitors. A supercapacitor can provide a higher charge-density than an electrolytic or tantalum capacitors, and can be useful for implementations that require delivery of bursts of current. In an example, the supercapacitor can be an electrochemical capacitor. In some examples, the supercapacitors can be used to supplement or replace power sources such as batteries, including Li+batteries, NiCd batteries, NiMH batteries, or other similar types of power sources. The measurement device can be configured to commence the measurement and/or diagnosis using the power stored in the energy-retaining component to perform the measurement and/or diagnosis.

According to the example systems, methods, and apparatus described herein, procedures and component activation sequences are provided that facilitate use of a measurement device for performing quantitative measurements as described herein. The measurement device may include no power source or a low-power source. The example procedures and component activation sequences also can be implemented in a measurement device or system that includes a relatively higher-power source. In such an implementation, the procedures and component activation sequences described herein can be implemented, for example, as a power-conserving technique.

In a non-limiting example, the example procedures and component activation sequences can be performed in conjunction with the energy harvesting described herein to implement a measurement device in performing a measurement and/or a diagnosis. The example component activation sequence can specify a sequence and timing of activation of specific components of the measurement device to facilitate the performance of a reliable measurement. The performance of the measurements may be made at any point after the activation is completed. Data indicative of the measurement performed, or information indicative of a diagnosis based on that measurement data, may be transmitted using a communication component and/or component protocol of the measurement device.

A measurement device can be configured such that it can be charged at substantially the same time that data is collected. The charge may be stored in, for example, a capacitor and/or battery of the system. The measurement device can be maintained in proximity to a computing device for a period of time that includes charging time and data pulling time. As a non-limiting example, the period of time can be about three seconds, about seven seconds, about ten seconds or about fifteen seconds. The boost converter takes a lot less power once it is charged. After this specified time period, other components of the system can be turned on.

In some implementations, data collected based on a measurement of the measurement device can be transmitted using a communication protocol to an external storage, a network, a server (e.g., of a data center), or a cloud database, including to a memory of an external device. For example, the communication protocol can be configured to transmit data via a wireless networks, a radio frequency communication protocol, Bluetooth® (including Bluetooth® low energy), near-field communication (NFC), and/or optically using infrared or non-infrared light-emitting-diode (LED). In other implementations, data collected based on a measurement of the measurement device can be stored to a memory of the measurement device for a period of time, and transferred (transmitted) at a later time to an external storage, a network, a server (e.g., of a data center), or a cloud database, including to a memory of an external device. In such implementations, the measurement device can be configured to store the data to local memory and reserve the right to use that option whether in a direct data transfer at the time of measurement or sometime afterwards.

As a non-limiting example, the measurement data can be made accessible to (with properly secured consent) medical doctors, health professionals, sports medicine practitioners, physical therapists, etc. For example, the system can be configured such that the patient, medical doctors, health professionals, sports medicine practitioners, physical therapists, etc. can get information indicative of the data measurement, metadata in connection with the data measurements (including an indication of when measurement was taken and/or when the data reading occurred), etc. In some implementations, the patient, medical doctors, health professionals, sports medicine practitioners, physical therapists, etc. can be given access to a graphical display or other analysis of the data measurements, such as, but not limited to, plots/charts/graphs of the measurement data.

The measurement devices of the present disclosure can be formed as a conformal sensor that is used for sensing, measuring, and/or otherwise quantifying at least one parameter, for example, of a sample. The systems, methods, and apparatuses of the present disclosure can use the results of analysis of data indicative of the at least one parameter for such applications as medical diagnosis, medical treatment, physical activity, sports, physical therapy and/or clinical purposes.

The procedures and component activation sequences described herein can be initiated dynamically on a computing device using processor-executable instructions configured as an application software program. The processor-executable instructions can include user instructions that specify to a user a sequence of steps for navigating the application software program.

In a non-limiting example, a device according to the principles described herein can be configured in a conformation, and in a form factor, that can indicate to a user the sequence of steps to follow for the desired component activation sequence. In an example, the application software program is configured to display instructions to a user to determine a proper placement of the measurement and/or diagnostic device relative to the computing device, and the indicate the length of time that the measurement and/or diagnostic device is to be maintained in that placement position to facilitate proper energy harvesting.

In some implementations of the measurement and/or diagnostic devices of the present disclosure, a user may write to the measurement and/or diagnostic device.

According to the example systems, methods, and apparatuses described herein, the measurement device can be coupled to a processor of the system that is configured to execute processor-executable instructions that facilitate performance of a measurement without a stable power source or a continual power source. The processor-executable instructions may be stored to a memory of the system. In an example, the processor may be configured to execute processor-executable instructions that execute procedures of an algorithm for ensuring a higher likelihood (probability) of a valid measurement using unstable power supply. In an example, the processor-executable instructions include instructions for performing error checking and/or self-monitoring to ensure the higher likelihood (probability) of a valid measurement. In an example, the system includes components that are configured to provide data caching and power caching.

The measurement device can include at least one power sub-circuit. The power sub-circuit can include at least one dynamic NFC enabled integrated circuit (NFC IC) coupled with a dual-interface, electrically erasable programmable memory (EEPROM). In other example implementations, other types of memories can be used, such as but not limited to a flash memory.

The NFC used in the IC herein can be configured based on a standard used, for example, for RFID tags and cell phones. In an example implementation, other forms of near field communication techniques can be used. For example, the measurement device could be configured to include a custom H-Field implementation that uses one or more custom tuned antennas and/or communication protocol.

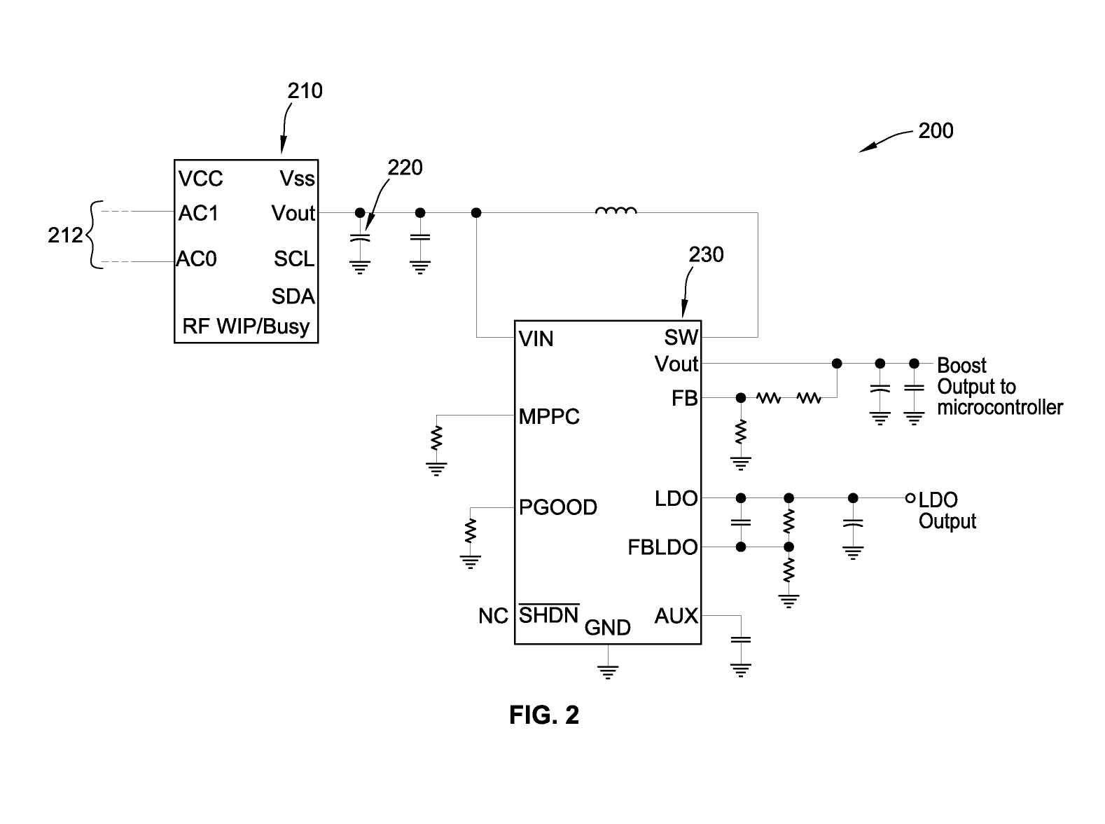

In some implementations, the NFC EEPROM is coupled to a DC-DC converter. Referring to FIG. 2, a power circuit 200 includes a wirelessly enabled energy harvesting device 210 (e.g., a near-field communication (NFC) enabled energy harvesting device such as an NFC EEPROM and/or an RFID component) used for storing energy in a storage capacitor 220. The wirelessly enabled energy harvesting device 210 is coupled to a DC-DC converter 230. The power circuit 200 can be included in the measurement device of the present disclosure for use in storing harvested energy. In operation, the wirelessly enabled energy harvesting device 210 voltage outputs to a low-leakage storage capacitor (e.g., the storage capacitor 220). As non-limiting examples, the storage capacitor 220 may be one or more of an aluminum electrolytic capacitor or an aluminum hybrid electrolytic capacitor. In some examples, the storage capacitors 220 has a capacity of about 470 microFarad (μF) or higher. In some examples, ultra-low leakage tantalum capacitors also can be used for the storage capacitor 220 if the measurement device is implemented for a measurement that does not draw much current and that can spare some amount of the leakage from the tantalum capacitors. In an example, the storage capacitor 220 serves as a reservoir for the wirelessly enabled energy harvesting device 210 in lieu of an on-board power source (e.g., the batteries 100a, 100b). In some example, the measurement device includes the DC-DC converter 230 that is a low input voltage model.

In some implementations, the wirelessly enabled energy harvesting device 210 is coupled to one or more optional antennas 212. The antennas 212 can be used to aid in wireless coupling of the wirelessly enabled energy harvesting device 210 with one or more wireless transmitting devices (e.g., an NFC transmitter, an RFID transmitter, a smartphone, and/or computing device 710 shown in FIG. 7) during a power harvesting operation.

In an example implementation, the characteristics of the DC-DC converter 230 are determined in order to determine the operation parameters of the measurement device including the same. For example, the capability of the power circuit 200 of the measurement device for energy harvesting can be determined based on the characteristics of the DC-DC converter 230. For example, to facilitate operation of the measurement device having no on-board power source or a low power source, the DC-DC converter 230 that draws low current across the wirelessly enabled energy harvesting device's 210 output voltage range can be selected. In other example implementations, any type of DC-DC converter can be used that has a low turn-on voltage and does not draw excessive current on starting-up. For instance, the DC-DC converter 230 can be a LT3105 converter (a converter that can operate using input voltages as low as about 225 mV). When the measurement device is positioned (including being held) relative to an external device to harvest energy, the initial output current can be limited. For example, when an external wireless transmitting device (e.g., a computing device) is disposed proximate to the measurement device that includes a wirelessly enabled energy harvesting device (e.g., an NFC EEPROM), the initial output current can be limited. If the entire circuitry of the measurement device draws power at this time, then the wirelessly enabled energy harvesting device cannot deliver the charge needed to startup or otherwise activate the circuit. For example, if portions of the circuitry that are configured for performing a medical diagnosis also draw power, then the wirelessly enabled energy harvesting device cannot deliver the charge needed to startup the circuit.

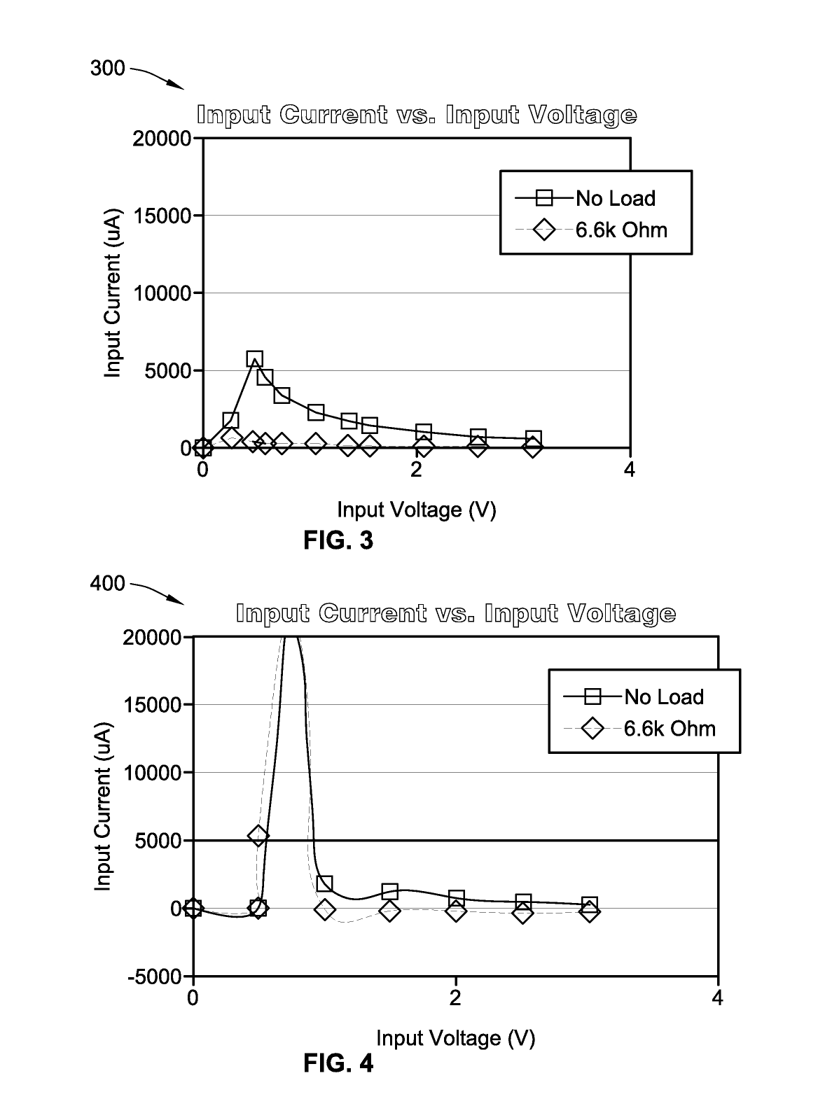

Referring to FIGS. 3 and 4, example plots 300, 400 of measurements of characteristics of example DC-DC converters (e.g., DC-DC converter 230) measured under no load and under a load of about 6.6 kOhms are shown. The characteristics are determined based on measurements of input current versus input voltage for the DC-DC converters. FIG. 3 shows an example high efficiency step-up DC-DC converter that can be operated from input voltages of about 225 mV, with a range of input voltages from about 225 mV to about 5V. FIG. 4 shows an example isolated DC-DC converter that takes input voltages of about 0.7V to about 5.5V.

As is evident by a comparison of FIGS. 3 and 4, the DC-DC converter measured in connection with the data shown in FIG. 3 draws relatively less current at low input voltages than the DC-DC converter measured in connection with the data shown in FIG. 4. Thus, the data illustrated in FIGS. 3 and 4 demonstrates the differences in current consumption of DC-DC converters at low voltages. It is evident from this data that DC-DC converters do not all behave the same way at low voltages, or even at startup. Thus, based on a characterization of the operational properties of DC-DC converters (e.g., using the plots 300, 400), a designer of a measurement device according to the present disclosure can choose a DC-DC converter that exhibits the optimal properties on startup. For example, the DC-DC converter associated with FIG. 3 would be a preferable choice (over the DC-DC converter associated with FIG. 4) for implementation in the measurement device of the present disclosure as it consumes relatively less current on startup.

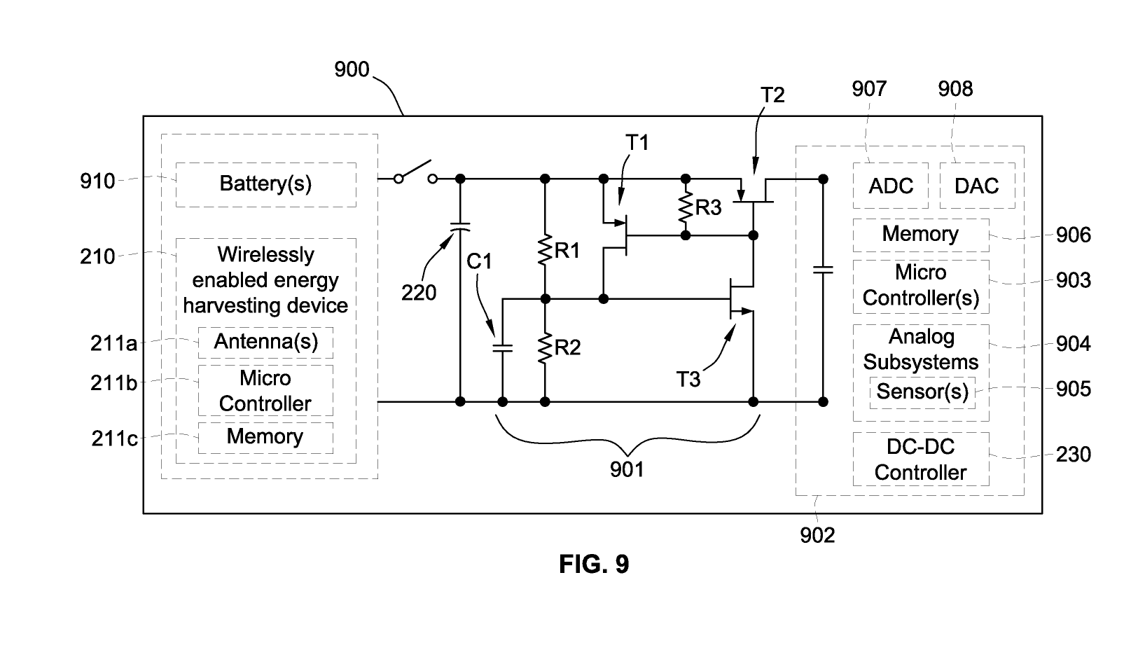

In an example implementation, a microcontroller and/or a timing control circuit (e.g., a pre-charge circuit 901 shown in FIG. 9) of the measurement device can be configured to execute processor-readable instructions to control the timing of the power sequencing of components of the system. In another example implementation, a microcontroller of the measurement device can be configured to execute processor-readable instructions to determine which sub-systems get power. In some implementations, the measurement device includes a microcontroller, a digital-to-analog converter (DAC), at least one amplifier, and at least one wirelessly enabled energy harvesting device (e.g., an NFC EEPROM), where each of the DAC, amplifier, and wirelessly enabled energy harvesting device has its own power supply and/or timing control from the microcontroller. In other example implementations, other types of data storage devices can be used, such as but not limited to a flash memory. In an example implementation, by separating power into each of the components (e.g., the DAC, amplifier, NFC EEPROM), the microcontroller can be configured to execute processor-readable instructions to exert granular control of the current consumption of the overall system. In an example implementation, the microcontroller can be configured to execute processor-readable instructions to change power usage dynamically. While these examples are described relative to microcontrollers, in other example systems with configurations that do not include microcontrollers, the processor of these example systems can be configured to execute these processor-readable instructions.

At least one processor unit and/or a timing control circuit (e.g., a pre-charge circuit) of the measurement device can be configured to execute processor-executable instructions to control the sequence of initiation of each sub-system drawing current from at least one wirelessly enabled energy harvesting device (e.g., an NFC EEPROM) and/or at least one storage capacitor of the measurement device. In an example implementation, the at least one wirelessly enabled energy harvesting device 210 of the system can be implemented to supply a set amount of current and voltage when an example computing device is brought in proximity to the measurement device (such as but not limited to a diagnostic device).

In some example implementations, the measurement device can be operated to draw an average current within the deliverable current of the wirelessly enabled energy harvesting device 210, but not to draw current continuously, continually, or consistently. For example, there can be surges of current consumption that can exceed the instantaneous amount of current the wirelessly enabled energy harvesting device 210 can deliver, such as, but not limited to, at startup of the measurement device.

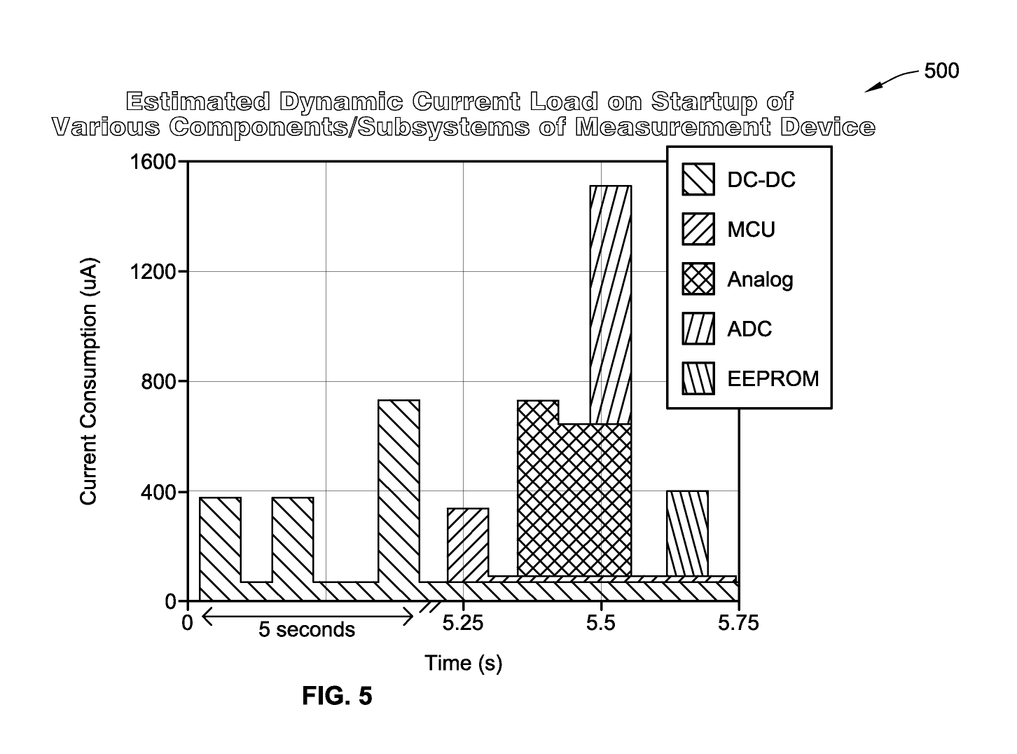

Referring to FIG. 5, a chart 500 illustrates current load of a measurement device of the present disclosure as various sub-systems are turned on. If all electronic components are turned on at the same time or substantially simultaneously, the current could be too much for the wirelessly enabled energy harvesting device 210 chip to deliver. FIG. 5 illustrates how the current may surge, as a function of time from start-up, when various sub-systems are turned on. If all the components of the system are turned on at the same time, the wirelessly enabled energy harvesting device 210 may be unable to deliver the current needed. In addition, the DC-DC converter 230 itself may require multiple current surges before it can run continuously. Based on data derived from characterization of the electronic properties of the DC-DC converter 230, it is determined that more power could gradually be drawn from the wirelessly enabled energy harvesting device 210 once the DC-DC converter 230 has successfully boosted the input signal to the regulated output voltage. The DC-DC converter 230, which draws current from the wirelessly enabled energy harvesting device 210 and the storage capacitor 220, draws a dynamic amount of current based on its input voltage and the current load. The current load is determined by how many subsystems (e.g., integrated circuits of the measurement device) are on. According to the principles described herein, by reducing the load initially, the DC-DC converter 230 can be caused to draw less current on start-up and not cause the output voltage of the wirelessly enabled energy harvesting device 210 to collapse.

In an example, based on a characterization of one or more components of a sub-system, a measurement device can be configured having no power source (or only a low-power source), that is powered using coupled wireless transmission of energy which is stored locally. The measurement device is operated using a specified sequence for powering up components of the sub-system(s) to avoid or substantially prevent current spikes and/or power spikes. Data from characterization of the powering up behavior of components of the sub-system, such as shown in FIG. 5, can be used to determine the specified sequence for powering up components. Once the measurement device is powered up, the measurement device can perform functions such as but not limited to data gathering, data storage and/or data transmission, as described herein.

In an example, the processor and/or a timing control circuit (e.g., a pre-charge circuit) executes processor-executable instructions that time the turning on or activation of components of the sub-system based on minimizing the overlapping current spikes that can occur with the turning on of each component. For example, based on data indicative of the dynamic current load on start-up of various components of the subsystem (such as shown in the example of FIG. 5), the timing of the turning on or activation of components of the sub-system can be determined. As shown in the non-limiting example of FIG. 5, the timing of the turning on or activation of components such as the DC-DC converter 230, microcontroller (MCU), analog components, analog-to-digital converter (ADC), and the NFC EEPROM 210, of the sub-system can be determined based on data indicative of the dynamic current load on start-up of these components. Non-limiting examples of the analog components include amplifiers, sensors, and multiplexers that are included in the analog circuits.

In an example implementation, at least one memory of the measurement device can be used to store any of the processor executable instructions described herein.

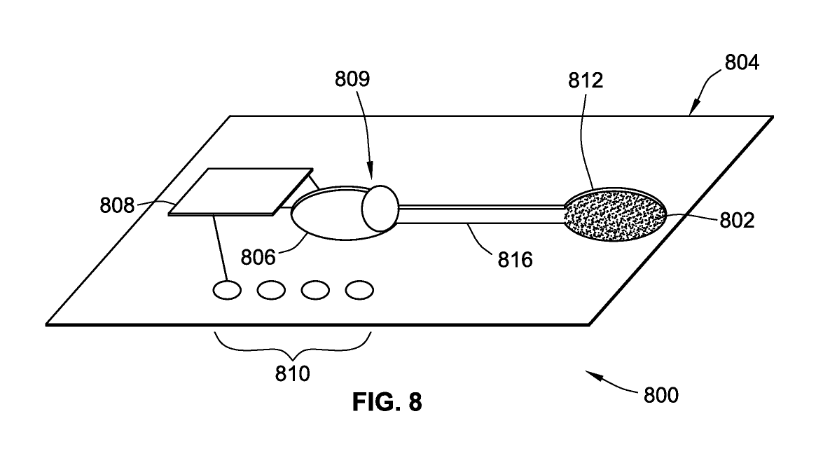

In an example implementation, once the DC-DC converter 230 is running continuously, other sub-systems (e.g., a functional circuit, such as, for example, an integrated circuit including a sensor for use in measuring an analyte concentration in a fluid sample) can be turned on or otherwise activated sequentially. For example, FIG. 6 illustrates an example sequence 600 of startup of components of a measurement device (e.g., measurement device 720, 800). At 602, a measurement device harvests power from a wireless transmitting device (e.g., a computing device, such as, for example, a smartphone with NFC and/or RFID capabilities, computing device 710) over time (e.g., over a time interval Tpower_delay, as monitored and/or indicated using a counter). At a time interval (Tpower_sequence_delay) greater than or about equal to a power-up sequence delay (as monitored and/or indicated using a counter), analog subsystems (e.g., a functional circuit including one or more analog and/or digital components, such as, for example, a sensor for use in measuring an analyte concentration of a fluid sample) of the measurement device (e.g., measurement device 800) are powered up using the power accumulated at 604. In some implementations, the analog subsystems and/or the components thereof (e.g., sensors, separate and distinct integrated circuits, etc.) are sequentially powered up in a predetermined sequence to minimize power consumption at startup and after the DC-DC converter 230 has settled. At 606, at least one sensor of the measurement device is excited (e.g., turned on/activated), and data from an analysis performed by the sensor or other portion of the measurement device is read (e.g., collected). For example, the data collection may be performed through iteration through one or more channels (e.g., channel 816 in FIG. 8) of the measurement device. At 608, the collected data is stored in a memory of the microprocessor unit; however, the collected data can alternatively and/or additionally be stored in a flash memory of the measurement device. In some implementations, the collected data may be stored in an external computing device, for example, by being transmitted using a communication interface and/or transmission protocol of the measurement device. At 610, a procedure of program readings onto the NFC IC is performed. For example, this can include checking the wirelessly enabled energy harvesting device 210 (e.g., an NFC integrated circuit, an NFC EEPROM, etc.) for valid data against the an MCU memory. At 612, once the checking is completed, the measurement device can be returned to a substantially dormant state (such as, but not limited to, a sleep mode), until it is powered up again, for example, using harvested energy and/or using an attached power source.

Another example implementation to control power sequencing is as follows. This example facilitates successful operation using energy solely from harvested energy from a wireless transmitting device (e.g., a computing device such as a smartphone) using the wirelessly enabled energy harvesting device 210 of the measurement device (e.g., the measurement device 800). That is, in this implementation, the measurement device lacks a power source. However, in implementations where the measurement device includes a power source, such as a low-power or higher power source, these power sources may be kept dormant or offline while the harvested energy is used according to the sequences described herein. Initially, a wireless transmitting device (e.g., a smartphone) is brought in proximity (e.g., within two inches) to the measurement device, whereupon the wirelessly enabled energy harvesting device 210 (e.g., an NFC EEPROM) begins to output current and voltage from its output pin(s) (e.g., Vout). As such, the storage capacitor 220 of the measurement device begins to charge. As the storage capacitor 220 begins to charge, the DC-DC converter 230 starts to boost the voltage. The load on the DC-DC converter 230 is from a microcontroller of, for example, a functional circuit drawing power/current therefrom. The processor can execute the processor-executable instructions to cause various analog sub-systems (e.g., one or more functional circuits) to begin to turn on after a period of time delay. The period of time delay can be determined based on based on characterization of the startup characteristics of each component or portion of the subsystem, as described herein. These sub-systems also can be characterized for dynamic current draw. The processor can execute the processor-executable instructions to cause a sequence of powering up of subsystem components such that the maximum dynamic current drawn at any given time is to a level that the wirelessly enabled energy harvesting device 210 can handle (i.e., below a maximum available current at the output of the wirelessly enabled energy harvesting device 210 at a given time). If the threshold is exceeded and the output voltage of the wirelessly enabled energy harvesting device 210 collapses, the microcontroller may execute processor-executable instructions to cause the startup sequence to be repeated, with a change in the amount of interval of time that the system waits between powering on various portions of the sub-system. This can ensure that more time is given to the wirelessly enabled energy harvesting device 210 to deliver current to the storage capacitor 220 and/or other storage capacitors. In some implementations, the system is configured to vary the onset of powering on of subsystem components based on the differing energy delivery profile of the computing device used for energy harvesting. Different computing devices may have different power delivery profiles that may change the rate of energy harvesting. The example system is configured such that the various measurement devices (such as, but not limited to, a diagnostic device) can work to perform the data gathering and/or data analysis reliably using the harvested energy.

Another implementation provides for controlling the disposition and/or position of a measurement device relative to a computing device to facilitate optimal energy harvesting by the measurement device from the computing device. For example, the methods, systems and apparatuses of the present disclosure can be implemented to determine the optimal distance and/or optimal angle of orientation between the measurement device and the computing device for the measurement device to derive enough power, for example, enough power to obtain measurement data and/or to analyze the data (e.g., for diagnosis). In another example, the methods, systems, and apparatuses of the present disclosure can be implemented to determine the optimal timing of how long to position the measurement device relative to/adjacent to the computing device by, for example, specifying a minimal period of time. The minimal period of time can be displayed on a display of the computing device (e.g., a smartphone) used to charge the measurement device. The harvested power can be used by the measurement device to receive delivery of data.

In an example, a processing unit of the computing device can be configured to execute processor-executable instructions (such as, but not limited to, software) to aid a user in the optimal placement/orientation of the computing device relative to the measurement/diagnosis device for the specified amount of time. In an example, the computing device can be configured to display to a user instructions for proper positioning of the computing device relative to the example measurement device. In another example, the computing device can be configured to display to a user an indication reaffirming the proper placement and/or duration of placement of the computing device relative to the example measurement device to ensure continuous or continual powering of the example measurement device.

As a non-limiting example, the duration of placement of the computing device relative to the measurement device for sufficient energy harvesting could last for about five seconds, about seven seconds, about ten seconds or about fifteen seconds. In an example, the placement duration is from about ten seconds to about fifteen seconds.

Referring to FIG. 7, a flow diagram 700 illustrating step-by-step instructions for placement of a wireless transmitting device 710 (e.g., a computing device such as a smartphone) relative to a measurement/diagnostic device 720 is shown. A processing unit (not shown) of the wireless transmitting device 710 can be configured to execute processor-executable instructions to display to a user on a display 712 of the wireless transmitting device 710 graphics depicting step-by-step instructions for placement of the wireless transmitting device 710 relative to the measurement device 720. The step-by-step instructions can be displayed as an animation. As shown in FIG. 7, the display 712, such as, but not limited to, a graphical user interface of the wireless transmitting device 710, displays instructions to a user to ensure proper placement of the wireless transmitting device 710 relative to the measurement device 720 and timing for maximum power harvesting. In some implementations, the wireless transmitting device 710 or the measurement device 720 provides an audible and/or visual indication when there is sufficient coupling for good harvesting energy and/or data transmission between the wireless transmitting device 710 and the measurement device 720. For example, the wireless transmitting device 710 or the measurement device 720 may be configured to sound an audible beep when there is good transmission. In another example, the wireless transmitting device 710 or the measurement device 720 may include at least one component that emits light or that causes the wireless transmitting device 710 to vibrate when there is a good connection for harvesting energy and/or data transmission established between the wireless transmitting device 710 and the measurement device 720. As a non-limiting example, the measurement device 720 can include at least one LED to emit light for such an indication and/or other indications. In another example, a portion of a display of the measurement device 720 may be caused to illuminate to provide the indication, such as, but not limited to, a display based on electronic ink.

In some implementations, a processing unit of the wireless transmitting device 710 executes processor-executable instructions to display a timer 714 that instructs the user as to how long to wait before disposing the wireless transmitting device 710 relative to the measurement device 720 and/or how long to hold the wireless transmitting device 710 relative to the measurement device 720. For example, where the measurement device 720 is used for sample analysis, the timing of how long to wait before disposing the wireless transmitting device 710 relative to the measurement device 720 can be specified based on an expected duration of the reaction between the sample and a chemical component (e.g., a reagent on the measurement device 720). This can be based on the time that it takes for a chemical reaction to occur between an analyte (e.g., glucose) in a bodily fluid (e.g., blood) and the chemical component (e.g., reagent). The wireless transmitting device 710 does not necessarily need to be disposed in the optimal position relative to the measurement device 720 during the time that the chemical reaction occurs. In such an example, the system can be configured such that the instructions to the user for proper placement can also indicate when the reaction is complete and analysis can begin. As another example, where the measurement device 720 is used for sample analysis, the timing of how long to hold the wireless transmitting device 710 in position relative to the measurement device 720 can be specified based on an expected duration of time for the various subsystems of the circuit to power on and/or an expected duration of time for the measurement device 720 to make the measurement.

In some implementations, processor-executable instructions (including a software application) of the wireless transmitting device 710 can be configured to work with processor-executable instructions (including a software application) of the measurement device 720 to maintain data integrity during transmission while the wireless transmitting device 710 is maintained in position relative to the measurement device 720. For example, a data cache can be included on a microcontroller of the measurement device 720; data can be delivered to the wirelessly enabled energy harvesting device (e.g., the same as, or similar to, the wireless enabled energy harvesting device 210) of the measurement device 710. In an example, there also can be an error check performed between the data received by the wireless transmitting device 710 and what is stored on the data cache of the microcontroller to validate the success of the file transfer, such as, but not limited to, the measurement data and/or any analysis of the measurement data. Parity checking can be performed at any available opportunity to ensure validity of the data.

In some implementations, in the event of failed transmission or poor power sequencing, processor-executable instructions (including a software application) of the computing device and processor-executable instructions (including a software application) of the measurement device 720 can be configured to use different time delays to sequence power as well as re-transmit data. These can increase the probability of successful acquisition of measurement data.

In any implementation disclosed herein, the disclosed system can be configured to run several supply rails and/or control lines for example, to separately power up and/or down various portions of the sub-systems (e.g., portions of a functional circuit) of the measurement device (e.g., 720, 800).

In any implementation disclosed herein, the disclosed system can be configured to independently control loads of various components of the measurement device (e.g., 720, 800), such as, but not limited to, a functional circuit of the measurement device and/or separate and distinct integrated circuits therein, the microcontroller, the amplifier(s), the digital to analog converters, the wirelessly enabled energy harvesting device 210, near-field communication (NFC) components, etc.

In any implementation disclosed herein, a processor of the disclosed system can be configured to execute processor executable instructions to implement a timed power-on sequence, to prevent simultaneous current spikes through two or more components of the measurement device (e.g., 720, 800). Such simultaneous current spikes could cause a load current surge that could disrupt the output voltage (e.g., Vout) of the wirelessly enabled energy harvesting device 210. In any implementation disclosed herein, a processor of the disclosed system can be configured to execute processor executable instructions to cause the disclosed system to recover if the load current is too heavy. For example, the processor can be configured to execute processor executable instructions to modify the timing of the power-on sequence of the components of the measurement device (e.g., 720, 800) if a startup failure occurs, such as, but not limited to, modifying the time interval of delay between power-on of some of the components.

The measurement device (e.g., measurement device 720, 800) can be used to analyze a sample of biological tissue, such as, but not limited to, blood. The data collected from the measurement device can be analyzed to detect the presence of, or lack thereof, certain nutrients in blood. For example, a sample of blood may be taken from a subject or from another stored source and be analyzed using an assay or other chemical present on, or introduced to, a measurement portion or sample receiving portion (e.g., receiver 812) of the measurement device. In another example, the sample may be processed prior to introduction to the measurement portion of the measurement device. A blood sample may be filtered to derive blood plasma and then the blood plasma can be introduced to the measurement portion of the measurement device. The data collected from the measurement device can be analyzed to detect HIV, malaria, or used to evaluate the level of cholesterol or of micronutrients, such as, but not limited to, iron, iodine, vitamin A levels, etc.

A measurement device according to the present disclosure may be configured as a low-cost glucose reader that does not need an on-board power source. A blood sample or a sample derived from blood may be introduced to a designated portion of the glucose reader that includes an analyte for a glucose level analysis. Electronic components (e.g., electronic circuitry and/or a functional circuit 808) of the glucose reader can be powered up and operated according to any of the example methods described herein using energy harvesting from a wireless transmitting device (e.g., wireless transmitting device 710) positioned (including being held) relative to the glucose reader. Processor-executable instructions (including application software) may be configured to provide an indication to a user when sufficient time has passed for the reaction analysis to be completed and/or when sufficient time has passed for the energy harvesting to have been completed. Furthermore, the data readout capability need not be integrated with the glucose reader device. Rather, in some implementations, the glucose reader transmits data, for example, using a communication protocol, to the wireless transmitting device or other data storage or when sufficient time has passed for a retrieval system. The glucose reader may be disposable or re-usable for a limited number of uses or for a limited period of time (e.g., for about two weeks or about a month, etc.). The low-cost, disposable glucose reader may include multiple channels (e.g., channel 816), each of which can be used to the analyze blood samples to provide a glucose level measurement.

In some implementations, a measurement device according to the present disclosure is a biomarker measurement device for detection of various types of biomarkers in a sample. The sample can be a blood sample, derived from blood samples (including plasma), other body fluid, secretion or excretion (including fecal matter or urine), or other tissue sample or tissue biopsy. Such a biomarker measurement device can be used for detection of a biomarker indicative of a condition, such as, but not limited to, a cardiac condition. Analysis of measurements could be used to indicate the onset of the cardiac condition, degree of progression of the cardiac condition, or quantifying the risk of mortality from the heart condition. The biomarker measurement device can be used for detection of a biomarker, such as, but not limited to, levels of the ST-2 protein. Measurements of the level of the ST-2 biomarker can be used to monitor heart failure onset or quantify a degree of progression of heart failure, including providing a measure of heart failure mortality. In some implementations, the biomarker measurement device is used to monitor or quantify the levels of biomarkers of inflammation, atherogenesis, endothelial function, thrombosis, ischemia, necrosis, hemodynamic stress, renal dysfunction, metabolic dysregulation, lipid dysregulation, or brain damage (see, e.g., Table 1 for a list of biomarkers that can be detected using the biomarker device and a corresponding condition indicated by the sensed/detected biomarker).

TABLE 1

| Condition | Example Biomarkers |
|---|---|
| brain damage | S100 beta |
| | neuron-specific enolase |
| Metabolic/lipid | adiponectin |
| dysregulation | resistin |
| | c-peptide |
| | cholesteryl ester transfer protein activity |
| Renal dysfunction | cystatin-C |
| | neutrophil gelatinase-associated lipocalin |
| | (NGAL) |
| Ischemia/necrosis | malondialdehyde-modified low-density |
| | lipoprotein |
| | Fatty acid binding protein |
| Hemodynamic stress | B-type natriuretic peptide (BNP) or |
| | N-terminal pro b-type natriuretic |
| | peptide (NT-proBNP) |
| | Urocortin-1 |
| | Endothelin-1 |
| Oxidative stress | Lp-PLA2 mass |
| | oxidized Apolipoprotein A1 |
| | Asymmetric dimethylarginine or other |
| | L-arginine metabolic product |
| Thrombosis | von Willebrand factor (vWF) |
| | Soluble CD40 ligand (sCD40L) |
| | Thrombus precursor protein (TpP) |
| Endothelial Function | E-selectin |
| Inflammation/atherogenesis | metalloproteinases |
| | (MMP-9, MMP-11) |
| | chemotactic molecules |
| | (MCP-1, CCR1, CCR2) |
| | Markers of fibrosis (galectin-3) |
| | Myeloid-related proteins 8/14 (MRP8/14) |

The biomarker measurement device can be used to measure various types of biomarkers in a sample indicative of neurological disorders. For example, the biomarker measurement device can be used to measure biomarkers for Parkinson's, schizophrenia, Huntington's disease, frototemporal dementia, multiple sclerosis, or a stroke.

In some implementations, the biomarker measurement device is used to quantify the levels of a biomarker, and based on an analysis thereof, an indication of a cardiac condition is derived. The analysis can be performed using a processor of the biomarker measurement device (e.g., measurement device 720) or using a processor of an external computing device (e.g., the wireless transmitting device 710). The sample may be introduced to a designated portion of the measurement device. The electronic components of the measurement device can be powered up and operated according to any of the example methods described herein using energy harvesting from a computing device positioned (including being held) relative to the measurement device. Processor-executable instructions (including an application software) may be configured to provide an indication to a user when sufficient time has passed for the reaction analysis to be completed and/or when sufficient time has passed for the energy harvesting to be completed. The biomarker measurement device may be configured to transmit data (including analysis of measurements), for example, using a communication protocol, to the computing device or other data storage or when sufficient time has passed for a retrieval system.

A biomarker measurement device of the present disclosure can be used to detection troponin levels in a sample. In such an implementation, the sample can be a blood sample or derived from a blood sample. Increased troponin levels, even merely a detectable amount, in the sample can serve as a biomarker of damage to heart muscle or a heart disorder, such as, but not limited to, myocardial infarction. For example, even small increases in troponin levels can serve as an indicator of cardiac muscle cell death. As a non-limiting example, this implementation can be used to determine whether chest pains are due to a heart attack. Using the biomarker measurement device, the troponin levels can be quantified, and based on an analysis of the measurements, a determination can be made whether the troponin levels are indicative of myocardial necrosis consistent with myocardial infarction. The analysis can be performed using a processor of the measurement device or using a processor of an external computing device. A blood sample or a sample derived from blood may be introduced to a designated portion of the measurement device. The electronic components of the measurement device can be powered up and operated according to any of the example methods described herein using energy harvesting from a computing device positioned (including being held) relative to the measurement device. Processor-executable instructions (including an application software) may be configured to provide an indication to a user when sufficient time has passed for the reaction analysis to be completed and/or when sufficient time has passed for the energy harvesting to have been completed. The measurement device may be configured to transmit data, for example, using a communication protocol, to the computing device or other data storage or when sufficient time has passed for a retrieval system.

A software application (App) can be provided for the wireless transmitting device (e.g., the wireless transmitting device 710) that causes the wireless transmitting device and/or the measurement device to provide visual and/or auditory instructions or prompts (including vibrational prompts) to a user. The visual and/or auditory instructions or prompts (including the vibrational prompts) to the user can be used to indicate the duration of time that the wireless transmitting device and the measurement device should be positioned (including being held) relative to each other before getting a data measurement, such as, but not limited to, a reading from an glucose reader, troponin level reader, or other biomarker measurement device. The visual and/or auditory instructions or prompts (including vibrational prompts) can be used to signal to the user a delay time for the chemical reactions of the analysis to complete before the computing device and the measurement device are positioned (including being held) relative to each other.

A software application (App) can be provided for the wireless transmitting device (e.g., the wireless transmitting device 710) that causes the wireless transmitting device and/or the measurement device to provide visual and/or auditory instructions or prompts (including vibrational prompts) to a user to indicate the degree of success in bringing the wireless transmitting device in position relative to the measurement device. For example, the visual and/or auditory instructions or prompts may be used to indicate a proximity of the wireless transmitting device to the measurement device. A miss-positioning of as little as a few centimeters could cause significantly reduced efficiency during the energy harvesting procedure.

The measurement device can be configured to provide visual and/or auditory indicators or signals (including vibrational prompts) to a user to aid in the placement of the measurement device relative to the wireless transmitting device. For example, the visual and/or auditory indicators or signals may change levels to indicate a degree of proximity of the wireless transmitting device to the measurement device. In an example, the visual and/or auditory indicators or signals could get brighter, louder, or stronger (as applicable) when the measurement device is close to the wireless transmitting device.

The measurement device may include at least one energy generating component to provide the energy for powering the subsystems (e.g., one or more functional circuits including, for example, one or more sensors). For example, the measurement device may include at least one photo-voltaic component, to generate power on exposure of the measurement device to electromagnetic energy (including solar energy). The energy generating component can be at least one solar micro-cell.

The measurement device can be configured such that energy can be introduced to the measurement device via a coupling of an audio port of the wireless transmitting device (e.g., the wireless transmitting device 710) to the measurement device. In this example, the power to the measurement device can be modulated, regulated, and/or otherwise optimized through use of volume control of the wireless transmitting device. For example, a software application (App) is provided for the computing device that causes the wireless transmitting device and/or the measurement device to change the volume control of the computing device to modulate, regulate and/or otherwise optimize the power transfer to the measurement device.

The measurement device can be configured such that energy can be introduced via a piezoelectric component or thermoelectric component coupled to the wireless transmitting device. For example, the measurement device may include a port that couples to the piezoelectric component or the thermoelectric component to facilitate the energy harvesting.

The measurement device can be configured as a RFID reader. At a point of interrogation, the RFID reader measurement device can be positioned relative to a computing device. With energy transfer to the RFID reader, portions of the system related to identification (ID) information (including an ID badge) and/or other sensor or measurement portions (including a temperature sensor) can be powered up and interrogated. Based on the sequential powering on of components as described herein, these portions of the systems can be run for several seconds, much longer than the millisecond timescales that may be required for solely RFID application. This can be beneficial, for example, in applications where the RFID reader is also configured for sample analysis. For example, taking readings for analyte measurement portions of the system could take time on the order of several seconds or longer if the analyte measurement portion is a multi-channel system for testing several channels of samples substantially simultaneously.

Any of the measurement devices of the present disclosure can be configured such that a capacitor or other low energy delivery component is integrated with an analyte reader of the measurement device. The analyte reader can be a glucophone glucose reader, a troponin level reader, or other biomarker measurement device.

According to some implementations of the present disclosure, a measurement device can be configured for providing quantitative information relating to a sample, where the measurement device includes a substrate that has at least one paper-based portion, a sample receiver at least partially formed in or disposed on a paper-based portion of the substrate, electronic circuitry (e.g., one or more functional circuits) and at least one indicator electrically coupled to the electronic circuitry. The electronic circuitry and the at least one indicator are at least partially formed in or disposed on the substrate. The electronic circuitry generates an analysis result based on an output signal from the sample or a derivative of the sample. The at least one indicator provides an indication of the quantitative information relating to the sample based at least in part on the analysis result.

Referring to FIG. 8, the measurement device 800 for providing quantitative information relating to a sample 802 is shown. The measurement device 800 includes a substrate 804, and a container 806 at least partially formed in or disposed on the substrate 804 to retain the sample 802. The container 806 can be, for example, a well or an indentation formed in the substrate 804. The container 806 can substantially enclose a space containing the sample 802, or have an open top. Electronic circuitry 808 integrated with or coupled to the substrate 804 is used to analyze an output signal from the sample 802, or from a derivative 809 of the sample 802 to provide an analysis result. The derivative can be an output from a reaction between the sample and a reagent, or results from a reaction within the sample 802 itself (e.g., when the sample 802 is subject to stimulation such as an electrical stimulation or an optical stimulation). The measurement device 800 also includes at least one indicator 810 integrated with or coupled to the substrate 804, and electrically coupled to the electronic circuitry 808 (e.g., one or more functional circuits), to provide the quantitative information relating to the sample 802 based at least in part on the analysis result. The indicator 810 is readable by a user, and thus serves as a human interface.

The measurement device 800 further includes a receiver 812 formed at least partially in or on the substrate 804 to receive the sample 802. The receiver 812 can be, for example, an indentation or an orifice in the substrate 804. A channel 816 is formed at least partially in or on the substrate 804 to transfer the sample 802 from the receiver 812 to the container 806. A drop of the sample 802, such as a drop of blood, once received by the receiver, can be drawn to the container 806 via the channel 816, by capillary action for example.

While the measurement device 800 is shown with a single channel 816, the measurement device 800 can be configured with two or more channels and/or capillaries. Any number of the channels and/or capillaries can be used for a single measurement or multiple measurements.

In some implementations, the substrate 804 includes a piece of paper for wicking the sample from the receiver 812 to the container 806 via a capillary action within the paper. As such, the channel 816 need not necessarily be carved out from the paper substrate; rather, the paper can be engineered, for example, by printing wax on the desired location of the channel, or imprinted or pressed to allow the capillary action to occur in preferred directions.

The substrate 804 can further include PDMS disposed over the paper-based portion. In one example, the PDMS is uncured. In another example, the substrate 804 further includes a urethane disposed over the piece of paper. The urethane can be UV curable.

In some implementations, the substrate 804 is ultrathin, for example, having a thickness on the order of approximately 200 microns or less. Such an ultrathin structure of the substrate 804 allows the entire measurement device 800 to be foldable.

In some implementations, the measurement device 800 includes a reagent retained in the container 806 to react with the sample 802. The output signal being analyzed by the electronic circuitry 808 indicates a reaction output of the reagent and the sample. The fluidic channel 816 transfers the sample 802 to the container 806 to react with the reagent, forming the derivative 809 being analyzed.

In some implementations, the fluidic channel 816 is formed between a piece of paper of the substrate 804 and a water resistant material of the substrate 804. In some such implementations, the substrate 804 is formed by bonding the piece of paper and the water resistant material together. In one example, the water resistant material includes PDMS.

In some implementations, the substrate 804 does not include paper. For example, in some such implementations, the substrate 804 is fabricated based on a variety of other materials, such as, but not limited to, glass, elastomer, parylene, plastic, polyimide, PDMS, or other polymer. In an example, the substrate 804 may be based on any thin composite material, including a composite material composed of woven fiberglass cloth with an epoxy resin binder, such as but not limited to FR4.

The measurement device 800 can be used to measure a variety of properties of the sample 802. For example, the quantitative information provided by the indicator 810 can be one of a glucose level, a T-cell concentration, a microorganism concentration, a bovine serum albumin (BVA) concentration, a bacterial concentration, a water-based pathogen concentration, a viral load, antibody level, antigen level, a diagnosis of malaria, tuberculosis or dengue fever, or cardiac enzyme concentration.

In some implementations, the measurement device 720 (FIG. 7) includes a power sub-circuit (e.g., the power sub-circuit shown in FIG. 2), the wirelessly enabled energy harvesting device 210, the storage capacitor 220, the DC-DC converter 230, a microcontroller, a digital-to-analog converter (DAC), an analog-to-digital converter (ADC), at least one amplifier, one or more analog devices (e.g., functional circuits, sensors, etc.), or any combination thereof.

In some implementations, the electronic circuitry 808 of the measurement device 800 (FIG. 8) includes one or more functional circuits, a power sub-circuit (e.g., the power circuit 200 shown in FIG. 2), a pre-charge circuit 901 (FIG. 9), a timing control circuit, a counter, the wireless enabled energy harvesting device 210, the storage capacitor 220, the DC-DC converter 230, a microcontroller, an digital-to-analog converter (DAC), an analog-to-digital converter (ADC), at least one amplifier, one or more analog devices (e.g., sensors), or any combination thereof.

Referring to FIG. 9, a circuit diagram 900 of a measurement device (e.g., measurement device 720, 800) according to aspects of the present disclosure includes an optional pre-charge circuit 901 electrically coupled between the storage capacitor 220 of the power circuit 200 of FIG. 2 (e.g., the energy storage component) and the DC-DC converter 230 (among other components) of the power circuit 200 of FIG. 2. In some implementations of the measurement devices of the present disclosure, the pre-charge circuit 901 is included into the power circuit 200 of FIG. 2 to (i) prevent electrical communication between the storage capacitor 220 and the DC-DC converter 230 until the storage capacitor 220 stores an amount of energy greater than a threshold energy level and (ii) maintain an electrical communication between the storage capacitor 220 and the DC-DC converter 230 thereafter. As such, the pre-charge circuit 901 aids in providing a stable power connection from a battery 910 (e.g., battery 100a, 100b) of the system and/or the wirelessly enable energy harvesting device 210 (e.g., which harvests energy from a wireless transmitting device in lieu of or in addition to the battery 910) to the DC-DC converter 230. In some implementations, R1 of the pre-charge circuit 901 has a resistance of 1 Mohm, R2 has a resistance of 10 Mohm, and R3 has a resistance of 1 Mohm. The pre-charge circuit 901 also includes three transistors T1, T2, T3. The pre-charge circuit 901 helps provide enough startup charge such that the DC-DC converter 230 starts reliably. The pre-charge circuit 901 includes a capacitor C1 which aids the pre-charge circuit 901 in performing a hardware based timing operation (e.g., the pre-charge circuit 901 includes timing control or a timing control circuit therein) of when to send current to, for example, the DC-DC controller 230.

According to some implementations, as shown in FIG. 9, the wirelessly enabled energy harvesting device 210 may include one or more antennas 211a, a microcontroller 211b, and one or more memories 211c. Further, a circuit portion 902 of the circuit 900 including the DC-DC converter 230 may also include one or more functional circuits including, for example, one or more microcontrollers 903, one or more analog subsystems 904 (e.g., one or more sensors 905 such as, for example, an analyte sensor for sensing one or more analyte concentrations in a fluid sample), one or more memories 906, an analog-to-digital converter 907, a digital-to-analog converter 908, or any combination thereof. In some implementations, the microcontroller 903 is configured to perform a timing operation (using hardware and/or software) of when to activate and/or supply power to, for example, the analog subsystems 904, the sensor 905, the memory 906, the ADC 907, the DAC 908, or any combination thereof. In such implementations, the microcontroller 903 is able to control the order and/or time for tuning on (i.e., activation) the other elements in the circuit portion (e.g., functional circuit), which helps minimize and/or prevent the components (e.g., the sensor 905) from draining the power supplied from the DC-DC converter 230 and/or the storage capacitor 220 in a manner that causes the device including the circuit 900 to fail, crash, or otherwise not perform as intended (e.g., conduct one or more measurement tests).

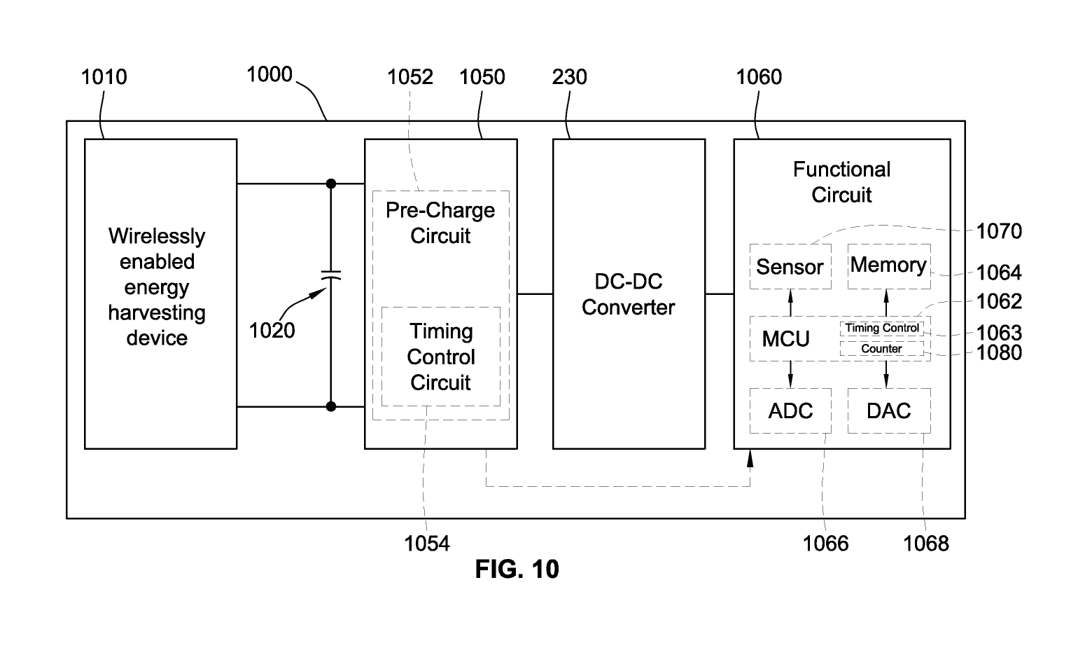

Referring to FIG. 10, where like reference numerals are used for like components described herein, a device 1000 (e.g., a measurement device) includes a wirelessly enabled energy harvesting device 1010, an energy storage device 1020, a control circuit 1050, a DC-DC converter 230, and a functional circuit 1060. The wirelessly enabled energy harvesting device 1010 is the same as, or similar to, the wirelessly enabled energy harvesting device 210 described herein. The wirelessly enabled energy harvesting device 1010 is configured to receive wireless signals from a wireless transmitting device (e.g., a smartphone enables with NFC) and convert and/or harvest those signals into energy. The harvested energy is stored in the energy storage device 1020, which can be a storage capacitor (e.g., the same as, or similar to, the storage capacitor 220). The energy storage device 1020 is electrically coupled between the wirelessly enabled energy harvesting device 1010 and the control circuit 1050, which is configured to control activation of other components of the device 1000, such as, for example, the DC-DC converter 230. In some alternative implementations, the control circuit 1050 is coupled with a counter (not shown) for providing an input to the control circuit 1050 for use in controlling the activation of one or more components of the device 1000. The control circuit 1050 is for determining activation of one or more components of the device 1000 (e.g., the DC-DC converter 230) by monitoring and waiting for a critical mass of charge to aggregate in the energy storage device 1020. After a specific amount of time, dictated by hardware component values and circuit(s), and after energy storage device 1020 reaches a certain voltage level, the path between the energy storage device 1020 and the DC-DC converter 230 becomes a closed circuit, thereby letting current flow to the DC-DC converter 230 from the energy storage device 1020. The control circuit 1050 can include a pre-charge circuit 1052 and/or a timing control circuit 1054. In some implementations, the pre-charge circuit 1052 includes the timing control circuit 1054 therein. The pre-charge circuit 1052 is the same as, or similar to, the pre-charge circuit 901 described herein and when included in the device 1000, ensures that the electrical connection between the energy storage device 1020 and the rest of the device components (e.g., the DC-DC converter 230 and the functional circuit 1060) is reliably established when the stored energy reaches a predetermined threshold as described herein. The timing control circuit 1054 controls the activation (e.g., turning on) of the DC-DC converter 230. Specifically, the timing control circuit 1054 works in conjunction with threshold detection on the energy storage device 1020 such that enough time passes for the energy storage device 1020 to build up charge. When the voltage of the energy storage device 1020 reaches a predetermined threshold, T3 turns on, turning on T2 in the pre-charge circuit 901, 1052, closing the circuit and letting current flow from the energy storage device 1020 to the DC-DC converter 230 via the control circuit 1050. The DC-DC converter 230 is electrically coupled to the control circuit 1050 and the functional circuit 1060. The DC-DC converter 230 receives a voltage output from the energy storage device 1020 and converts the received voltage output to a second voltage level to provide power to one or more components of the device 1000. Essentially, the DC-DC converter 230 steps up the voltage output of the energy storage device 1020 to a second higher voltage. The functional circuit 1060 is electrically coupled to the DC-DC converter 230 for receiving power therefrom. The functional circuit 1060 can include one or more micro-control units (MCU) 1062, one or more memory devices 1064, one or more analog-to-digital converters 1066, one or more digital-to-analog converters 1068, one or more sensors 1070, or any combination thereof. In some implementations, the functional circuit 1060 includes sufficient digital and/or analog components (e.g., integrated circuits) to determine an analyte concentration in a fluid sample. In some implementations, the MCU 1062 includes a timing control or timing control state machine 1063 that is configured to control the activation (e.g., turning on) of the various other components of the functional circuit 1060. For example, the MCU 1062 and/or the timing control 1063 performs a timing operation (using hardware and/or software) of when to activate and/or supply power to, for example, the memory 1064, the ADC 1066, the DAC 1068, the sensor 1070, or any combination thereof. In such implementations, the MCU 1062 and/or the timing control 1063 is able to control the order and/or time for tuning on (i.e., activation) the other elements in the functional circuit 1060, which helps minimize and/or prevent the components (e.g., the sensor 1070) from draining the power supplied from the DC-DC converter 230 and/or the storage capacitor 1020 in a manner that causes the device 1000 to fail, crash, or otherwise not perform as intended (e.g., conduct one or more measurement tests). In some implementations, the MCU 1062 and/or the timing control 1063 includes a counter 1080 for providing an input to the timing control 1063.

In some alternative implementations, instead of the MCU 1062 and/or the timing control 1063 controlling the functional circuit 1060, the control circuit 1050 is electrically connected with one or more components in the functional circuit 1060 for controlling (e.g., activating) the components by, for example, activating one or more switches (e.g., transistors) within the functional circuit 1060

In some implementations, the functional circuit includes an MCU 1062, a memory device 1064, an ADC 1066, and a sensor 1070. In some such implementations, the control circuit 1050 is configured to activate (i.e., turn on) the DC-DC converter 230, which powers the MCU 1062. Then the MCU 1062 and/or the timing control circuit 1063 is configured to activate (i.e., turn on) the memory device 1064, the ADC 1066, and the sensor 1070 at predetermined times and in a predetermined sequence to, for example, ensure that the device 1000 starts-up properly with all components therein sufficiently powered.

While various implementations have been described throughout the present disclosure, it is contemplated that any element, component, circuit, device, etc. described in reference to one implementation and/or figure can be included in any other implementation. For example, the pre-charge circuit 901 can be included in any device of the present disclosure. For another example, the functional circuit 1060 can be included in any implementation of the present disclosure. For yet another example, the counter 1080 can be included in any implementation of the present disclosure.

### ALTERNATIVE IMPLEMENTATIONS

Implementation 1.

A device comprising: a wirelessly enabled energy harvesting component; an energy storage component electrically coupled to the wirelessly enabled energy harvesting component for storing energy harvested by the wirelessly enabled energy harvesting component; and a functional circuit for performing a measurement, the functional circuit being coupled to the energy storage component such that the functional circuit is powered solely by the energy harvested by the wirelessly enabled energy harvesting component and stored in the energy storage component.

Implementation 2.

The device of implementation 1, wherein the energy harvested by the wirelessly enabled energy harvesting component is from a wireless transmitting device positioned adjacent to the device.

Implementation 3.

The device of implementation 2, wherein the wireless transmitting device is a smart phone.

Implementation 4.

The device of implementation 1, further comprising a DC-DC converter electrically coupled to the energy storage component for receiving a voltage output from the energy storage component and converting the received voltage output to a second voltage level to provide power to functional circuit and/or one or more other components of the device.

Implementation 5.

The device of implementation 4, wherein the one or more other components includes a processor.

Implementation 6.

The device of implementation 4, wherein the one or more other components includes a controller.

Implementation 7.

The device of implementation 4, wherein the one or more other components includes a memory.

Implementation 8.

The device of implementation 4, wherein the one or more other components includes an analog-to-digital converter.

Implementation 9.

The device of implementation 4, wherein the one or more other components includes an digital-to-analog converter.

Implementation 10.

The device of implementation 4, wherein the one or more other components includes a sensor.

Implementation 11.

The device of implementation 4, wherein the one or more other components includes an analyte sensor for measuring an analyte concentration in a fluid sample.

Implementation 12.

The device of implementation 11, wherein the analyte is glucose and the fluid sample is blood.

Implementation 13.

The device of implementation 7, wherein the memory is a near-field communication electrically erasable programmable memory (NFC EEPROM) memory.

Implementation 14.

The device of implementation 4, wherein the one or more components of the measurement device includes at least two components that receive at least a portion of the power provided by the DC-DC converter at predetermined times in a predetermined sequence.

Implementation 15.

The device of implementation 4, wherein the one or more components of the measurement device and the DC-DC converter each receives at least a portion of the voltage output from the energy storage component at predetermined times in a predetermined sequence.

Implementation 16.

The device of implementation 15, further comprising a microcontroller for controlling a power-up sequence of the one or more components of the measurement device and the DC-DC converter according to the predetermined times and the predetermined sequence.

Implementation 17.

The device of implementation 1, further comprising a pre-charge circuit electrically coupled to the energy storage component, the pre-charge circuit being configured to (i) prevent electrical communication between the energy storage component and the functional circuit until the energy storage component stores an amount of energy greater than a threshold energy level and (ii) maintain an electrical communication between the energy storage component and the functional circuit thereafter.

Implementation 18.

The device of implementation 4, further comprising a pre-charge circuit electrically coupled between the energy storage component and the DC-DC converter, the pre-charge circuit being configured to (i) prevent electrical communication between the energy storage component and the DC-DC converter until the energy storage component stores an amount of energy greater than a threshold energy level and (ii) maintain an electrical communication between the energy storage component and the DC-DC converter thereafter.

Implementation 19.

The device of implementation 1, wherein the device is batteryless such that the energy storage component and the functional circuit are each powered solely by energy harvested by the wirelessly enabled energy harvesting component.

Implementation 20.

The device of implementation 4, wherein the device is batteryless such that the energy storage component, the DC-DC converter, and the functional circuit are each powered solely by energy harvested by the wirelessly enabled energy harvesting component.

Implementation 21.

The device of implementation 1, wherein the device lacks a battery.

Implementation 22.

The device of implementation 1, wherein the wirelessly enabled energy harvesting component includes a near-field communication (NFC) antenna.

Implementation 23.

The device of implementation 1, wherein the wirelessly enabled energy harvesting component includes an RFID antenna.

Implementation 24.

The device of implementation 1, wherein the wirelessly enabled energy harvesting component includes a near-field communication NFC antenna.

Implementation 25.

The device of implementation 24, wherein the NFC antenna is a coil.

Implementation 26.

The device of implementation 4, wherein the DC-DC converter is powered solely by energy harvested by the wirelessly enabled energy harvesting component.

Implementation 27.

The device of implementation 1, further comprising a communication interface for transmitting data from the device to a second device.

Implementation 28.

The device of implementation 27, wherein the second device is a wireless transmitting device.

Implementation 29.

The device of implementation 28, wherein the wireless transmitting device is a smartphone.

Implementation 30.

The device of implementation 29, wherein the smartphone includes a software application running thereon for communicatively connecting in a bidirectional manner to the device.

Implementation 31.

The device of implementation 1, wherein the device is a measurement device.

Implementation 32.

The device of implementation 1, wherein the device is a blood glucose measurement device.

Implementation 33.

The device of implementation 1, wherein the device is an analyte measurement device.

Implementation 34.

The device of implementation 1, further comprising a counter.

Implementation 35.

The device of implementation 1, further comprising a timing control circuit.

Implementation 36.

The device of implementation 1, wherein the energy storage component is a capacitor.

Implementation 37.

The device of implementation 1, further comprising a control circuit.

Implementation 38.

The device of implementation 37, wherein the control circuit is a pre-charge circuit.

Implementation 39.

The device of implementation 37, wherein the control circuit is a timing control circuit.

Implementation 40.

The device of implementation 37, wherein the control circuit is configured to control activation of other components of the device.

Implementation 41.

The device of implementation 37, wherein the other components of the device include the functional circuit.

Implementation 42.

The device of implementation 37, wherein the other components of the device include a DC-DC converter.

Implementation 43.

The device of implementation 37, wherein the other components of the device include a sensor.

Implementation 44.

The device of implementation 37, wherein the other components of the device include a processor and/or a controller.

Implementation 45.

The device of implementation 37, wherein the other components of the device include an analog-to-digital converter.

Implementation 46.

The device of implementation 37, wherein the other components of the device include a digital-to-analog converter.

Implementation 47.

The device of implementation 1, wherein the wirelessly enabled energy harvesting component is an NFC EEPROM.

Implementation 48.

The device of implementation 1, wherein the wirelessly enabled energy harvesting component is an RFID component.

Implementation 49.

A measurement device comprising: a near-field communication (NFC) enabled energy harvesting device; an energy storage component electrically coupled to the NFC enabled energy harvesting device for storing energy harvested by the NFC enabled energy harvesting device from an NFC transmitting device positioned adjacent to the measurement device; a DC-DC converter electrically coupled to the energy storage component; a counter; and a functional circuit electrically coupled to the DC-DC converter, wherein the energy storage component harvests and stores at least a portion of the energy harvested by the NFC enabled energy harvesting device until a first time T₁ set by the counter, wherein the DC-DC converter is activated at a second time T₂ set by the counter using at least a portion of the energy stored in the energy storage component, and wherein the functional circuit is activated at a third time T₃ set by the counter using at least a portion of the power provided by the DC-DC converter.

Implementation 50.

The device of implementation 49, wherein the functional circuit includes one or more components for performing a measurement.

Implementation 51.

The device of implementation 49, further comprising an NFC antenna coupled to the NFC enabled energy harvesting device.

Implementation 52.

The device of implementation 51, wherein the NFC enabled energy harvesting device comprises an NFC enabled erasable programmable memory (EEPROM).

Implementation 53.

The device of implementation 49, wherein the energy storage component is a storage capacitor or a supercapacitor.

Implementation 54.

The device of implementation 49, further comprising a timing control circuit coupled to the functional circuit, the timing control circuit being configured to cause the functional circuit to be activated at the third time T₃ using at least a portion of the power provided by the DC-DC converter.

Implementation 55.

The device of implementation 54, wherein the functional circuit comprises at least one sensor, and wherein the timing control circuit is configured to cause the sensor to be activated to perform a measurement at a fourth time T₄, which is after the third time T₃.

Implementation 56.

A measurement device comprising: a near-field communication (NFC) enabled energy harvesting device; an energy storage component electrically coupled to the NFC enabled energy harvesting device for storing energy harvested by the NFC enabled energy harvesting device from an NFC transmitting device positioned adjacent to the measurement device; a pre-charge circuit electrically coupled to the energy storage component; a DC-DC converter electrically coupled to the pre-charge circuit; and a functional circuit electrically coupled to the DC-DC converter, wherein the pre-charge circuit is configured to prevent electrical communication between the energy storage component and the DC-DC converter until the energy storage component stores an amount of energy greater than a threshold energy level and to maintain the electrical communication between the energy storage component and the DC-DC converter thereafter; and wherein the functional circuit is configured to activate using at least a portion of the power provided by the DC-DC converter.

Implementation 57.

The device of implementation 56, wherein the functional circuit includes one or more components for performing a measurement.

Implementation 58.

The device of implementation 56, further comprising an NFC antenna coupled to the NFC enabled energy harvesting device.

Implementation 59.

The device of implementation 58, wherein the NFC enabled energy harvesting device comprises an NFC enabled erasable programmable memory (NFC EEPROM).

Implementation 60.

The device of implementation 56, wherein the energy storage component is a storage capacitor or a supercapacitor.

Implementation 61.

The device of implementation 56, further comprising: at least one processing unit coupled to the functional circuit; and at least one memory to store processor-executable instructions, the at least one processor being communicatively coupled to the at least one memory, wherein, upon execution of the processor-executable instructions, the at least one processor: activates prior to the functional circuit using at least a portion of the power provided by the DC-DC converter; and causes the functional circuit to activate.

Implementation 62.

The device of implementation 61, wherein the functional circuit comprises at least one sensor, wherein upon execution of the processor-executable instructions, the at least one processor activates the sensor to perform a measurement at time subsequent to the functional circuit activating.

Implementation 63.

A measurement device for measuring an analyte in a fluid sample, the measurement device comprising: a wirelessly enabled energy harvesting device; an energy storage component electrically coupled to the wirelessly enabled energy harvesting device for storing energy harvested by the wirelessly enabled energy harvesting device from a wireless transmitting device positioned adjacent to the measurement device; a DC-DC converter electrically coupled to the energy storage component for receiving a voltage output from the energy storage component and converting the received voltage output to a second voltage level to provide power to one or more components of the measurement device; and a functional circuit for measuring a quantity of the analyte in the fluid sample, the functional circuit being coupled to the DC-DC converter such that the functional circuit obtains at least a portion of the power provided by the DC-DC converter.

Implementation 64.

The device of implementation 63, wherein the one or more components of the measurement device includes at least two components that receive at least a portion of the power provided by the DC-DC converter at predetermined times in a predetermined sequence.

Implementation 65.

The device of implementation 63, wherein the one or more components of the measurement device and the DC-DC converter each receives at least a portion of the voltage output from the energy storage component at predetermined times in a predetermined sequence.

Implementation 66.

The device of implementation 65, further comprising a microcontroller for controlling a power-up sequence of the one or more components of the measurement device and the DC-DC converter according to the predetermined times and the predetermined sequence.

Implementation 67.

The device of implementation 63, further comprising a pre-charge circuit electrically coupled between the energy storage component and the DC-DC converter, the pre-charge circuit being configured to (i) prevent electrical communication between the energy storage component and the DC-DC converter until the energy storage component stores an amount of energy greater than a threshold energy level and (ii) maintain an electrical communication between the energy storage component and the DC-DC converter thereafter.

Implementation 68.

The device of implementation 63, wherein the measurement device is batteryless such that the energy storage component, the DC-DC converter, and the functional circuit are each powered solely by energy harvested by the wirelessly enabled energy harvesting device.

Implementation 69.

The device of implementation 63, wherein the wirelessly enabled energy harvesting device includes a near-field communication (NFC) antenna, an RFID antenna, or both.

Implementation 70.

The device of implementation 63, wherein the wirelessly enabled energy harvesting device includes a near-field communication NFC antenna, the NFC antenna being a coil.

Implementation 71.

The device of implementation 63, wherein the DC-DC converter is powered solely by energy harvested by the wirelessly enabled energy harvesting device.

Implementation 72.

The device of implementation 63, wherein the one or more components of the measurement device include a communication interface for transmitting data from the measurement device to a second device.

Implementation 73.

The device of implementation 72, wherein the second device is the wireless transmitting device.

Implementation 74.

The device of implementation 63, wherein the wireless transmitting device is a smartphone including a software application running thereon for communicatively connecting in a bidirectional manner to the measurement device.

Implementation 75.

A measurement device comprising: a wirelessly enabled energy harvesting device; an energy storage component electrically coupled to the wirelessly enabled energy harvesting device for storing energy harvested by the wirelessly enabled energy harvesting device from a wireless transmitting device positioned adjacent to the measurement device; a counter; and a functional circuit electrically coupled to the energy storage component, wherein the energy storage component harvests and stores at least a portion of the energy harvested by the wirelessly enabled energy harvesting device until a first time T₁ set by the counter, and wherein the functional circuit is activated at a second time T₂ set by the counter using at least a portion of the energy store din the energy storage component.

Implementation 76.

The device of implementation 75, further comprising a DC-DC converter electrically coupled to the energy storage component and the functional circuit.

Implementation 77.

The device of implementation 76, wherein the DC-DC converter is activated at a third time T₃ set by the counter using at least a portion of the energy stored in the energy storage component.

Implementation 78.

The device of implementation 77, wherein the second time T₂ is greater than the third time T₃ and the third time T₃ is greater than first time T₁.

Implementation 79.

A measurement device comprising: a wirelessly enabled energy harvesting device; an energy storage component electrically coupled to the wirelessly enabled energy harvesting device for storing energy harvested by the wirelessly enabled energy harvesting device from a wireless transmitting device positioned adjacent to the measurement device; a pre-charge circuit electrically coupled to the energy storage component; a DC-DC converter electrically coupled to the pre-charge circuit; and a functional circuit electrically coupled to the DC-DC converter, wherein the pre-charge circuit is configured to prevent electrical communication between the energy storage component and the DC-DC converter until the energy storage component stores an amount of energy greater than a threshold energy level and to maintain the electrical communication between the energy storage component and the DC-DC converter thereafter; and wherein the functional circuit is configured to activate using at least a portion of the power provided by the DC-DC converter.

Implementation 80.

The device of implementation 1, wherein the measurement performed by the functional circuit is a measurement of a concentration of a substance in a fluid sample.

Implementation 81.

The device of implementation 80, wherein the device is flexible and stretchable.

Implementation 82.

The device of implementation 80, wherein the device is configured to be worn directly on skin of a user of the device.

Implementation 83.

The device of implementation 82, wherein the fluid sample is directly received by the device from the user.

Implementation 84.

The device of implementation 80, wherein the substance being measured is an analyte, a virus, a protein, bacteria, an enzyme, a toxin, or any combination thereof.

Implementation 85.

The device of implementation 80, wherein the fluid sample is blood, sweat, urine, saliva, tear drops, air, or any combination thereof.

Implementation 86.

The device of implementation 84, wherein the toxin is a mercury, lead, metal, plastic, carbon monoxide, or any combination thereof.

It is contemplated that any element or any portion thereof from any of implementations 1-86 above can be combined with any other element or elements or portion(s) thereof from any of implementations 1-86 to form an implementation of the present disclosure.

What is claimed is:

1. A measurement device comprising:
a near-field communication (NFC) enabled energy harvesting device;
an energy storage component electrically coupled to the NFC enabled energy harvesting device for storing energy harvested by the NFC enabled energy harvesting device from an NFC transmitting device positioned adjacent to the measurement device;
a DC-DC converter electrically coupled to the energy storage component;
a counter; and
a functional circuit electrically coupled to the DC-DC converter
wherein the energy storage component harvests and stores at least a portion of the energy harvested by the NFC enabled energy harvesting device until a first time T₁ set by the counter,
wherein the DC-DC converter is activated at a second time T₂ set by the counter using at least a portion of the energy stored in the energy storage component, and
wherein the functional circuit is activated at a third time T₃ set by the counter using at least a portion of the power provided by the DC-DC converter.

2. The device of claim 1, wherein the functional circuit includes one or more components for performing a measurement.

3. The device of claim 1, further comprising an NFC antenna coupled to the NFC enabled energy harvesting device.

4. The device of claim 3, wherein the NFC enabled energy harvesting device comprises an NFC enabled erasable programmable memory (EEPROM).

5. The device of claim 1, wherein the energy storage component is a storage capacitor or a supercapacitor.

6. The device of claim 1, further comprising a timing control circuit coupled to the functional circuit, the timing control circuit being configured to cause the functional circuit to be activated at the third time T₃ using at least a portion of the power provided by the DC-DC converter.

7. The device of claim 6, wherein the functional circuit comprises at least one sensor, and wherein the timing control circuit is configured to cause the sensor to be activated to perform a measurement at a fourth time T₄, which is after the third time T₃.

8. A measurement device comprising:
a near-field communication (NFC) enabled energy harvesting device;
an energy storage component electrically coupled to the NFC enabled energy harvesting device for storing energy harvested by the NFC enabled energy harvesting device from an NFC transmitting device positioned adjacent to the measurement device;
a pre-charge circuit electrically coupled to the energy storage component;
a DC-DC converter electrically coupled to the pre-charge circuit; and
a functional circuit electrically coupled to the DC-DC converter, wherein the pre-charge circuit is configured to prevent electrical communication between the energy storage component and the DC-DC converter until the energy storage component stores an amount of energy greater than a threshold energy level and to maintain the electrical communication between the energy storage component and the DC-DC converter thereafter; and
wherein the functional circuit is configured to activate using at least a portion of the power provided by the DC-DC converter.

9. The device of claim 8, wherein the functional circuit includes one or more components for performing a measurement.

10. The device of claim 8, further comprising an NFC antenna coupled to the NFC enabled energy harvesting device.

11. The device of claim 10, wherein the NFC enabled energy harvesting device comprises an NFC enabled erasable programmable memory (NFC EEPROM).

12. The device of claim 8, wherein the energy storage component is a storage capacitor or a supercapacitor.

13. The device of claim 8, further comprising:
at least one processing unit coupled to the functional circuit; and
at least one memory to store processor-executable instructions, the at least one processor being communicatively coupled to the at least one memory, wherein, upon execution of the processor-executable instructions, the at least one processor:
activates prior to the functional circuit using at least a portion of the power provided by the DC-DC converter; and
causes the functional circuit to activate.

14. The device of claim 13, wherein the functional circuit comprises at least one sensor, wherein upon execution of the processor-executable instructions, the at least one processor activates the sensor to perform a measurement at time subsequent to the functional circuit activating.

15. A measurement device for measuring a concentration of a substance in a fluid sample, the measurement device comprising:
a wirelessly enabled energy harvesting device;
an energy storage component electrically coupled to the wirelessly enabled energy harvesting device for storing energy harvested by the wirelessly enabled energy harvesting device from a wireless transmitting device positioned adjacent to the measurement device;
a DC-DC converter electrically coupled to the energy storage component for receiving a voltage output from the energy storage component and converting the received voltage output to a second voltage level to provide power to one or more components of the measurement device; and
a functional circuit for measuring a concentration of the substance in the fluid sample, the functional circuit being coupled to the DC-DC converter such that the functional circuit obtains at least a portion of the power provided by the DC-DC converter.

16. The device of claim 15, wherein the one or more components of the measurement device includes at least two components that receive at least a portion of the power provided by the DC-DC converter at predetermined times in a predetermined sequence.

17. The device of claim 15, wherein the one or more components of the measurement device and the DC-DC converter each receives at least a portion of the voltage output from the energy storage component at predetermined times in a predetermined sequence.

18. The device of 17, further comprising a microcontroller for controlling a power-up sequence of the one or more components of the measurement device and the DC-DC converter according to the predetermined times and the predetermined sequence.

19. The device of claim 15, further comprising a pre-charge circuit electrically coupled between the energy storage component and the DC-DC converter, the pre-charge circuit being configured to (i) prevent electrical communication between the energy storage component and the DC-DC converter until the energy storage component stores an amount of energy greater than a threshold energy level and (ii) maintain an electrical communication between the energy storage component and the DC-DC converter thereafter.

20. The device of claim 15, wherein the measurement device is batteryless such that the energy storage component, the DC-DC converter, and the functional circuit are each powered solely by energy harvested by the wirelessly enabled energy harvesting device.

21. The device of claim 15, wherein the wirelessly enabled energy harvesting device includes a near-field communication (NFC) antenna, an RFID antenna, or both.

22. The device of claim 15, wherein the wirelessly enabled energy harvesting device includes a near-field communication NFC antenna, the NFC antenna being a coil.

23. The device of claim 15, wherein the DC-DC converter is powered solely by energy harvested by the wirelessly enabled energy harvesting device.

24. The device of claim 15, wherein the one or more components of the measurement device include a communication interface for transmitting data from the measurement device to a second device.

25. The device of claim 24, wherein the second device is the wireless transmitting device.

26. The device of claim 15, wherein the wireless transmitting device is a smartphone including a software application running thereon for communicatively connecting in a bidirectional manner to the measurement device.

27. The device of claim 15, wherein the measurement device is flexible and stretchable and configured to be worn directly on skin of a user.

28. The device of claim 27, wherein the fluid sample is directly received by the measurement device from the user.

29. The device of claim 15, wherein the substance being measured is an analyte, a virus, a protein, bacteria, an enzyme, a toxin, or any combination thereof.

30. The device of claim 15, wherein the fluid sample is blood, sweat, urine, saliva, tear drops, air, or any combination thereof.
